(19) United States
(12) Patent Application Publication
(10) Pub. No.: US 2020/0061112 A1
(43) Pub. Date: Feb. 27, 2020

(54) Dendritic Cells as a Novel Delivery System for Immunotherapy

(71) Applicant: UNIVERSITY OF CONNECTICUT, FARMINGTON, CT (US)

(72) Inventors: PRAMOD K. SRIVASTAVA, AVON, CT (US); HAKIMEH EBRAHIMI-NIK, NEW BRITAIN, CT (US)

(21) Appl. No.: 16/612,481

(22) PCT Filed: May 10, 2018
(86) PCT No.: PCT/US2018/031953

§ 371 Date: Nov. 11, 2019

Related U.S. Application Data
(60) Provisional application No. 62/504,291, filed on May 10, 2017.

(51) Int. Cl. A61K 35/15; A61P 37/06; A61P 35/00; A61P 31/12; C07K 16/28; C12N 5/0784

(52) U.S. Cl. CPC A61K 35/15; A61P 37/06; A61P 35/00; A61P 31/12; A61K 45/06; C07K 16/2812; C07K 16/2818; C12N 5/0639; C07K 16/2815

(57) ABSTRACT

Described herein are isolated dendritic cells, e.g., blood or bone marrow derived dendritic cells, which can be combined with a neoepitope for immunotherapy.

### FIELD OF THE DISCLOSURE

The present disclosure is related to immunotherapy and more specifically delivery systems for immunotherapy.

### BACKGROUND

Harnessing the immune system to combat tumors has provided a vital new means—beyond traditional surgery, radiation, and chemotherapy—of treating and potentially curing cancer. Most approaches require immune recognition of neo-epitopes, which are novel antigens created by unique mutations arising in tumors. In addition to reducing immune-inhibiting checkpoint signals such as those mediated by CTLA-4, effective immunization against neo-epitopes typically requires effective antigen presentation by professional antigen presenting cells (APCs), such as dendritic cells (DCs). However, despite more than 20 years of preclinical investigations and clinical trials using DC-based vaccines, the optimal method for generating DC based vaccines for tumor rejection remains unclear.

The majority of studies evaluating DC vaccines efficacy have measured induced T cell responses. This focus is reasonable, since antigen-specific T cell responses are required for effective tumor rejection. However, there is not always a direct correlation between neo-epitope-specific T cell responses measured in vitro and tumor rejection in vivo.

What is needed are methods of identifying DCs that provide the best anti-tumor response, and compositions and methods that use the identified DCs.

### BRIEF SUMMARY

In an aspect, described herein is an isolated CD11c⁺ MHCII^{lo} CD11b^{hi}, CD24^{lo}, CD40⁻, CD86^{lo} dendritic cell, or an isolated population of such cells. In another aspect, described herein is an isolated CD11c⁺ MHCII^{lo/int} CD11b^{hi}, CD24^{-/lo}, CD40^{-/lo}, CD86^{-/lo} dendritic cell, or an isolated population of such cells.

In an aspect, described herein is an isolated dendritic cell, or an isolated population of dendritic cells, expressing at least 1, 2, 3, 4, 5, 6, 7, 8, 9 or 10 (e.g., at least 2 or at least 3) of the following markers as indicated: CD91^{hi}, LOX1^{hi}, TLR2^{hi}, CD80^{-/lo}, CD36^{lo}, CD209a^{-/lo}, mannose receptor C-type 1^{hi}, macrophage scavenger receptor 1^{hi}, TLR1^{hi}, TLR6^{hi}, and TLR7^{hi}, optionally in addition to CD11c⁺ MHCII^{lo/int}, or optionally in addition to CD11c⁺ MHCII^{lo/int} CD11b^{hi}, or optionally in addition to CD11c⁺ MHCII^{lo/int} CD11b^{hi} CD24^{-/lo}, CD40^{-/lo}, CD86^{-/lo}.

In an aspect, described herein is an isolated dendritic cell, or an isolated population of dendritic cells, expressing at least 2, 3, 4, 5, 6, 7, 8, 9 or 10 (e.g., at least 3) of the following markers as indicated: CD91^{hi}, LOX1^{hi}, TLR2^{hi}, CD80^{-/lo}, CD36^{lo}, CD209a^{-/lo}, mannose receptor C-type 1^{hi}, macrophage scavenger receptor 1^{hi}, TLR1^{hi}, TLR6^{hi}, TLR7^{hi}, C1s1^{hi}, Cfb^{hi}, Fos^{hi}, Hp^{hi}, Il18^{hi}, Il1a^{hi}, Il19^{hi}, Serpine1^{hi}, Serpinf1^{lo}, Il18^{hi}, Il1a^{hi}, Il1f9^{hi}, Il1rl1^{lo}, Ccr7^{lo}, Cd40^{lo}, Cd83^{lo}, Fgfr1^{lo}, Fscn1^{lo}, H2-DMb2^{lo}, H2-Oa^{lo}, H2-Ob^{lo}, H2-Aa^{lo}, H2-Ab1^{lo}, H2-Ea-ps^{lo}, H2-Eb1^{lo}, Il18^{hi}, Il1a^{hi}, Il1f9^{hi}, Jak2^{lo}, Stat4^{lo}, Fyn^{lo}, Itgae^{lo}, Mylk^{lo}, Ptk2^{lo}, Tln2^{hi}, Tlr9^{lo}, Tspan2^{lo}, and Ppp1r14^{lo}, optionally in addition to CD11c⁺ MHCII^{lo/int}, or optionally in addition to CD11c⁺ MHCII^{lo/int} CD11b^{hi}, or optionally in addition to CD11c MHCII^{lo/int} CD11b^{hi} CD24^{-/lo}, CD40^{-/lo}, CD86^{-/lo}. In any of the foregoing embodiments, the cells are human cells, in which the MHCII protein is an HLA (Human Leukocyte Antigen) such as HLA DR (Human Leukocyte Antigen—antigen D Related).

In another aspect, a composition comprises an isolated CD11c⁺ MHCII^{lo} CD11b^{hi}, CD24^{lo}, CD40⁻, CD86^{lo} dendritic cell derived from the blood or bone marrow of a cancer patient, and a neoepitope peptide or nucleic acid molecule encoding the neoepitope peptide, wherein the neoepitope peptide is specific to a tumor from the cancer patient.

In another aspect, a composition comprises an isolated population of CD11c⁺ MHCII^{lo} CD11b^{hi}, CD24^{lo}, CD40⁻, CD86^{lo} dendritic cells derived from the blood or bone marrow of a cancer patient, and a neoepitope peptide or nucleic acid molecule encoding the neoepitope peptide, wherein the neoepitope peptide is specific to a tumor from the cancer patient.

In yet another aspect, an immunotherapy method comprises administering the above composition to the cancer patient.

In another aspect, a composition comprises an isolated CD11c⁺ MHCII^{lo/int} CD11b^{hi}, CD24^{-/lo}, CD40^{-/lo}, CD86^{-lo} dendritic cell, or any other cell or population of cells described herein, derived from the blood or bone marrow of a subject, and a neoepitope peptide or nucleic acid molecule encoding the neoepitope peptide, wherein the neoepitope peptide is specific to a tumor from a cancer patient, wherein the subject is the cancer patient or a healthy subject.

In yet another aspect, an immunotherapy method comprises administering the above composition to the cancer patient.

In another aspect, a method of producing an immunotherapeutic composition comprises isolating a population of dendritic cells described herein from a subject's peripheral blood mononuclear cells or bone marrow cells, wherein the subject can be a cancer patient, based on the expression of markers identified herein in the dendritic cells. The method of producing an immunotherapeutic composition may include: (i) obtaining cells (e.g., monocytes or bone marrow cells) from a blood or bone marrow sample of a subject, (ii) optionally differentiating the obtained cells toward dendritic cells (e.g., using a method known in the art), (iii) isolating a population of dendritic cells described herein (such as based on the expression of markers identified herein, e.g., using FACS sorting), and (iv) combining (e.g., by way of pulsing) the isolated population of dendritic cells with a neoepitope peptide or nucleic acid molecule encoding the neoepitope peptide, wherein the neoepitope peptide is specific to a tumor from a cancer patient. In a specific embodiment, the step of isolating a population of dendritic cells is based on the expression of at least 1, 2, 3, 4, 5, 6, 7, 8, 9 or 10 or more (e.g., at least 3, at least 5, or at least 7) of the markers listed in any of Tables 1-4 (e.g., listed in Tables 2-4) as shown for P6 cells (immature dendritic cells). For example, where a biomarker listed in one or more of Tables 1-4 is shown to be expressed at an increased level in immature dendritic cells relative to mature dendritic cells, the immature dendritic cells of interest can be isolated based on such increased expression. In another example, where a biomarker listed in one or more of Tables 1-4 is shown to be expressed at a decreased level in immature dendritic cells relative to mature dendritic cells, the immature dendritic cells of interest can be isolated based on such decreased expression. The foregoing applies to any one marker, any combination of markers, or all markers listed in Tables 1-4. In a more specific example, the step of isolating a population of dendritic cells is based on the expression of at least 2, 3, 4, 5, 6, 7, 8, 9 or 10 of the following markers as indicated: CD91^{hi}, LOX1^{hi}, TLR2^{hi}, CD80^{-/lo}, CD36^{lo}, CD209a^{-/lo}, mannose receptor C-type 1^{hi}, macrophage scavenger receptor 1^{hi}, TLR1^{hi}, TLR6^{hi}, and TLR7^{hi}, optionally in addition to CD11c⁺ MHCII^{lo/int}, or optionally in addition to CD11c⁺ MHCII^{lo/int} CD11b^{hi}, or optionally in addition to CD11c⁺ MHCII^{lo/int} CD11b^{hi} CD24^{-/lo}, CD40^{-/lo}, CD86^{-/lo}. In a specific embodiment, described herein is a method of isolating monocytes or bone marrow cells, optionally differentiating the obtained cells toward dendritic cells, and isolating a population of cells having the biomarker expression as described herein for the immature dendritic cells described herein by separating such cells based on such biomarker expression (e.g., by FACS sorting for expression of any or all of the biomarkers described herein as characterizing the immature dendritic cell population).

In another aspect, a method of producing an immunotherapeutic composition comprises

isolating monocytes from a cancer patient's peripheral blood mononuclear cells or obtaining bone marrow cells from the cancer patient, differentiating the monocytes or the bone marrow cells toward dendritic cells using, for example, GM-CSF, IL-4, or both GM-CSF and IL-4,

FACS sorting the differentiated cells based on MHCII and D11c expression, and

isolating from the sorted cells a population of cells with the same phenotype as a mouse CD11c⁺ MHCII^{lo} CD11b^{hi}, CD24^{lo}, CD40⁻, CD86^{lo} GM-CSF derived dendritic cell, and

pulsing the population of cells with the same phenotype as a mouse CD11c⁺ MHCII^{lo} CD11b^{hi}, CD24^{lo}, CD40⁻, CD86^{lo} derived dendritic cells with a patient tumor neoepitope peptide or a nucleic acid encoding a neoepitope peptide from a cancer patient to provide the immunotherapeutic composition, wherein the subject is the cancer patient or a healthy subject.

In another aspect, a method of producing an immunotherapeutic composition comprises

isolating monocytes from a subject's peripheral blood mononuclear cells or obtaining bone marrow cells from the subject, differentiating the monocytes or the bone marrow cells toward dendritic cells using, for example, GM-CSF, 11-4, FLT-3L, or both GM-CSF and IL-4,

FACS sorting the differentiated cells based on MHCII and CD11c expression, and

isolating from the sorted cells a population of CD11c⁺ MHCII^{lo/int} CD11b^{hi}, CD24^{-lo}, CD40^{-/lo}, CD86^{-/lo} dendritic cells, or any other population of cells described herein, and

pulsing the CD11c⁺ MHCII^{lo/int} CD11b^{hi}, CD24^{-/lo}, CD40^{-/lo}, CD86^{-/lo} dendritic cells, or any other isolated population of cells, with a tumor neoepitope peptide or a nucleic acid encoding a neoepitope peptide from a cancer patient to provide the immunotherapeutic composition, wherein the subject is the cancer patient or a healthy subject.

In a specific embodiment, provided herein is a method of isolating monocytes or bone marrow cells as described above, differentiating the isolated cells as described above, and isolating a population of cells having the biomarker expression as described herein for the immature dendritic cells described herein by separating such cells based on such biomarker expression (e.g., by FACS sorting for expression of any or all of the biomarkers described herein as characterizing the immature dendritic cell population as described herein).

In another aspect, a method of producing an immunotherapeutic composition comprises:
isolating monocytes from a subject's peripheral blood mononuclear cells or obtaining bone marrow cells from the subject,

differentiating the monocytes or the bone marrow cells toward dendritic cells using, for example, GM-CSF, IL-4, FLT-3L, or both GM-CSF and IL-4,

isolating from the differentiated cells a population of cells expressing: (i) at least 3 of the following markers as indicated: CD91^{hi}, LOX1^{hi}, TLR2^{hi}, CD80^{-/lo}, CD36^{lo}, CD209a^{-/lo}, mannose receptor C-type 1^{hi}, macrophage scavenger receptor 1^{hi}, TLR1^{hi}, TLR6^{hi}, and TLR7^{hi}, (ii) at least 7 of the following markers as indicated: CD91^{hi}, LOX1^{hi}, TLR2^{hi}, CD80^{-/lo}, CD36^{lo}, CD209a^{-/lo}, mannose receptor C-type 1^{hi}, macrophage scavenger receptor 1^{hi}, TLR1^{hi}, TLR6^{hi}, TLR7^{hi}, CD11c⁺ MHCII^{lo/int} CD11b^{hi}, CD24^{-/lo}, CD40^{-/lo}, CD86^{-/lo}, or (iii) at least 2 of the following markers as indicated: CD91^{hi}, LOX1^{hi}, TLR2^{hi}, CD80^{-/lo}, CD36^{lo}, CD209a^{-/lo}, mannose receptor C-type 1^{hi}, macrophage scavenger receptor 1^{hi}, TLR1^{hi}, TLR6^{hi}, and TLR7^{hi}, and all of the following markers: CD11c⁺ MHCII^{lo/int} CD11b^{hi}, and

pulsing the isolated population of dendritic cells with a tumor neoepitope peptide or a nucleic acid encoding a neoepitope peptide from a cancer patient to provide the immunotherapeutic composition, wherein the subject is the cancer patient or a healthy subject.

In a specific embodiment, the isolating of a population of cells is performed by one or more steps of FACS sorting based on the expression of the marker(s).

### BRIEF DESCRIPTION OF THE DRAWINGS

FIGS. 1A-E show immunization with BMDCs as a delivery system of Neo1 protects mice better against MethA tumor growth in comparison with splenocytes and bone marrow derived macrophages (BMDMs). For all tumor growth graphs, the Y- and X-axis display average tumor size (mm²) and days post tumor challenge, respectively. FIG. 1A. BALB/c mice were immunized twice, one week apart, with either splenocytes pulsed with DMSO as control, 15×10⁶ splenocytes, 3×10⁶ GMCSF-derived BMDCs or 3×10⁶ BMDM; pulsed with 100 μM Neo1 followed by 75 μg 9D9 treatment given at second immunization and every three days after tumor challenge, or without 9D9 treatment. Seven days after the second immunization, they were challenged with 95K MethA and tumor growth was measured. Each line is an indicator of tumor growth in a single mouse. FIG. 1B. BALB/c mice were immunized with 3×10⁶ FLT3-derived BMDCs either pulsed with DMSO or 100 uM Neo1. The tumor challenge and 9D9 treatment was the same as part A. FIG. 1C. BALB/c mice were immunized with 3×10⁵ monocytes GM-CSF IL-4-derived DCs either pulsed with DMSO or 100 uM Neo1. The tumor challenge and 9D9 treatment was the same as part A. FIG. 1D. Total TCI scores group average for FIG. 1A data set. The X-axis shows group numbers and the Y-axis represents total TCI scores. FIG. 1E. BALB/c mice were injected with 250 μg of ISo, CD8 or CD4 depletion antibodies 2 days before each immunization and tumor challenge and every week afterwards. 9D9 treatment was the same as part A.

FIGS. 2A-E show Neo1 pulsed BMDCs work as both a reservoir and an antigen presenter. For all the left panel graphs, the Y- and X-axis display average tumor size (mm²) and days post tumor challenge, respectively. Tumor growth was measured twice a week. Each line is an indicator of tumor growth in a single mouse. All the right panels show total TCI scores for the indicated groups. FIG. 2A. BALB/c mice were immunized with BALB/c BMDCs pulsed with DMSO or Neo1 followed by 75 μg 9D9 treatment given at second immunization and every three days after tumor challenge or without 9D9. Seven days after the second immunization they were challenged with 95K MethA. FIG. 2B. BALB/c mice were immunized with C57BL/6J BMDCs pulsed with DMSO or Neo1. 9D9 treatment and tumor challenge was the same as part A. FIG. 2C. C57BL/6J mice were immunized with β2Microglobulin^{-/-} C57BL/6J BMDCs pulsed with either DMSO or LP SIINFEHL. Seven days after the second immunization, they were challenged with 150K B16-OVA F0. FIG. 2D. Normalized TCI score for the mentioned groups. FIG. 2E. OT-I CD8⁺ T cells were labeled with 5 μM CFSE (Biolegend) and about 5×10⁵ labeled OT-I CD8⁺ T cells were adoptively transferred into two groups (n=3) of β2M^{-/-} mice, on day −3. After 24 hours, mice of the control group and experimental group were intradermally immunized with BM DCs alone or BMDCs pulsed with longer version of SIINFEKL, respectively. Draining lymph nodes were harvested from individual mice, on day 0 (72 hrs after OT-I transfer) and dilution of CFSE in gated CD45. 1⁺ OT-I CD8⁺ T cells was analyzed by flow cytometry.

FIGS. 3A and B show different subpopulations of BMDCs have different tumor rejection capacities. FIG. 3A. The phenotype of GM-CSF derived BMDCs cultures at day 7. BMDCs were sorted to three different subpopulations based on MHC II and CD11c expression as follow “P7” MHCII⁻ CD11c⁻, “P6” MHCII^{lo} CD11c⁺ and “P5” MHCII^{hi} CD11c⁺. P5 and P6 are divided further based on the CD11b expression (P6: MHCII^{lo} CD11b^{hi} and P5: MHCII^{hi} CD11b^{lo/int}). Boxes represent gates and percentage of cells in each gate. Histograms indicate surface expression of the indicated markers by P5 and P6 subsets. FIG. 3B. The top panel shows tumor growth in BALB/cJ mice that-were immunized with 500K of either whole BMDCs, P5, P6 or P7 pulsed with Neo1 or DMSO as control. All the immunizations were performed twice, one week apart with or without 9D9 treatment. Seven days after the second immunization, the mice were challenged with 95K MethA and tumor growth was measured. Each line is an indicator of tumor growth in a single mouse. The middle panel shows the group average of tumor growth. The bottom panel represents total TCI score for each group.

FIGS. 4A-E show that BMDCs are the most potent adjuvants. For all tumor growth graphs, each line represents tumor growth in a single mouse (n=5 per group). FIG. 4A. BALB/cJ mice were immunized twice, one week apart, with 100 μM Neo1-pulsed splenocytes (1.5×10⁷), GMCSF-BMDCs (3×10⁶) or BMDM (3×10⁶). Mice were administered 75 μg 9D9 antibody as indicated in Methods of Example 4. Seven days after the second immunization, mice were challenged with 95,000 Meth A cells, and tumor growth was measured. FIGS. 4B, 4C. BALB/cJ mice were immunized with 100 μM Neo1-pulsed 3×10⁶ FLT3L-BMDCs (FIG. 4B) or Mo-DCs (FIG. 4C). Other details were the same as in FIG. 4A. FIG. 4D. Total TCI scores for FIGS. 4A, 4B and 4C data sets with 9D9 are shown. TCI scores of GM-CSF- and FLT3-BMDCs were statistically higher than BMDM and splenocytes (GM-CSF-BMDCs/BMDM P=0.003, GM-CSF-BMDC/splenocytes P=0.0162, FLT3-BMDCs/BMDM P=0.015 and FLT3-BMDCs/splenocytes P=0.043). FIG. 4E. BALB/c mice were injected with 250 μg of CD8 or CD4 depletion antibodies (or isotype controls) as described in Methods (Example 4). Tumor challenge was the same as in FIG. 4A. Experiments in FIG. 4 were repeated between two to ten times, with the exception of FIG. 4C, which was done only once. This experiment was not repeated since obtaining enough blood to isolate monocytes required over 60 mice per group.

FIGS. 5A-5E show that BMDCs act as ADCs as well as APCs. FIG. 5A. BALB/cJ mice were immunized with Neo1-pulsed BALB/cJ or C57BL/6 BMDCs followed by 9D9 treatment, and tumor challenge as in FIG. 4. Tumor growth was measured (n=10 per group). FIG. 5B. C57BL/6 mice were immunized with LP SIINFEHL-pulsed β2M^{-/-} C57BL/6J BMDCs. Seven days after the second immunization, mice were challenged with 150,000 B16-OVA F0 tumor cells. Tumor growth was measured (n=5 per group). FIGS. 5C and 5D represent normalized TCI score for FIGS. 5A and 5B. The average TCI score of BALB/cJ mice immunized with BALB/cJ BMDCs was significantly higher (P=0.0001) than BALB/cJ mice immunized with C57BL/6 BMDCs. FIG. 5E. CFSE-labeled OT-I CD8+ T cells were adoptively transferred into two groups (n=3 per group) of β2M^{-/-} mice, on day −3. After 24 h, all mice were immunized intradermally with BMDCs alone or LP SIINFEHL-pulsed BMDCs. Draining lymph nodes were harvested from individual mice 72 h after OT-I transfer and dilution of CFSE on CD45.1+ gated OT-I CD8+ T cells was analyzed. The percentage of OT-I CD8 T cell proliferation in β2M^{-/-} mice immunized with β2M^{+/+} BMDC was significantly higher (P<0.0001) than in the control group. Experiments in FIGS. 5A-5D were done in whole or in parts, at least twice.

FIGS. 6A-6G show that subpopulations of BMDCs have distinct tumor rejection capacities. FIG. 6A. The phenotype of GM-CSF-BMDCs cultures at day 7. CD11c⁺MHCII⁺ BMDCs (FIG. 6A, left) were divided further based on the CD11b expression (FIG. 6A, right; P6: MHCII^{lo} CD11b^{hi} and P5: MHCII^{hi} CD11b^{lo/int}). Boxes represent gates and percentage of cells in each gate. Histograms indicate surface expression of the indicated markers by P5 and P6 subsets (left peak isotype stained, right peak antibody stained). FIG. 6B shows the photomicrograph of P5, P6 and P7 cell sub-populations (200×). FIG. 6C shows tumor growth in BALB/cJ mice immunized with Neo1-pulsed 500,000 un-fractionated BMDCs, P5, P6 or P7 cells. All the immunizations were performed twice, one week apart with 9D9 treatment. Mice were challenged with Meth A cells and tumor growth monitored as in FIG. 4 (n=5 per group). FIG. 6D shows the group average of tumor growth for FIG. 6C. FIG. 6E shows area under the curve (AUC) scores for each group of FIG. 6C. The AUC was calculated from day 6-31 since all groups showed uniform growth from days 0-6. The AUC value for P6 population was significantly lower than that of P5 (P=0.029), P7 (P=0.0005), total BMDC (P=0.0012) and the control group (P<0.0001). FIG. 6F. Photomicrographs of P5, P6 and P7 sub-populations that were incubated with FITC microbeads. The right and middle panels represent the side scattered images of the cells in the bright and dark field, respectively. The right panel shows the FITC channel. The number of the beads taken up by each sub-population is indicated at the top right corner of the image. FIG. 6G. Cells were incubated with FITC and acquired by MACS Quant® machine. X-axis and Y-axis represent the FITC channel and count, respectively. Experiments in FIGS. 6A-6G were done at least two times.

FIG. 7 shows a heat map of the significant pathways selected for the P5 and P6 cell sub-populations using the IPA tools. IPA identified 869 up- and down-regulated genes in P6 compared to P5, eligible for pathways analysis. Canonical pathways were identified and analyzed from the IPA libraries. P values <0.05 was used to define differentially expressed genes. This experiment was performed once.

FIGS. 8A and 8B show the phenotype of BALB/cJ splenocytes (FIG. 8A) and bone marrow derived macrophages (BMDM) (FIG. 8B). Splenocytes and BMDM were characterized using antibody markers for macrophages, DCs, B cells, CD4 and CD8 T cells. Boxes represent gates and percentages of cells in each gate.

FIG. 9 shows the results of incubation of sorted P5, P6 and P7 sub-populations with FITC microspheres. P5, P6 and P7 sub-populations were incubated with 0.5 micron FITC microspheres for 30 minutes to test the capacity of antigen uptake of each sub-population. Using an Image Stream®X Mark II Imaging Flow Cytometer, the number of beads in each sub-population were quantified. The X-axis shows the number of beads and the Y-axis indicates the percentage of the cells. R3 gate is representative of the cells that were not able to take up any beads. R4 shows the percentage of the cells that took up 1-3 beads, and R5 represents the percentage of the cells that had more than 3 beads. The statistics for each graph are shown below each graph.

The above-described and other features will be appreciated and understood by those skilled in the art from the following detailed description, drawings, and appended claims.

### DETAILED DESCRIPTION

The inventors used a tumor rejection assay to determine the optimal antigen-presenting cell vaccine approach for mediating anti-tumor effects, and sought to better define the mechanisms by which these cells act. To model approaches amenable to clinical translation, the inventors evaluated DCs that were developed from monocytes, which could then be pulsed with immunogenic neoepitopes. Granulocyte-macrophage colony-stimulating factor (GM-CSF)-induced, bone marrow-derived dendritic cells (BMDCs) provided the best anti-tumor response, and these cells mediated more rapid tumor rejection than did FMS-like tyrosine kinase-3 (FLT-3)-derived BMDCs. The BMDCs were further characterized, leading to the finding that a subpopulation that was CD11c and MHC class II low (MCHII^{lo}), or CD11c⁺ and MHC class II low/intermediate (MCHII^{lo/int}), was most effective in mediating tumor regression. Surprisingly, tumor rejection was dependent on both antigen presentation and reservoir function of BMDCs.

Currently, the optimal tumor vaccine strategy remains a subject of intense debate, especially for tumor mutation-derived neoepitopes. Different types of adjuvants have been tested in cancer therapy vaccines such as mineral adjuvants and cytokines, RNA based adjuvants, liposomes, tensoactive agents and bacterial products. But none of them had efficient tumor rejection capacity in clinical trials. Dendritic cell (DC)-based vaccines function well in preclinical models, but less so in clinical trials, and the optimal DC subset for tumor vaccines remains unclear. This challenge can be considered in three parts. The first is the identification of tumor-specific antigens, typically mutation-induced neoepitopes that can mediate immune recognition. The second is the development of an antigen delivery system, whether cell-based, adjuvant-based, or both, that efficiently presents the neoepitopes to the immune system in a manner that mediates an effective immune response. Finally, steps must be taken to overcome the immunosuppressive tumor microenvironment, such as is achieved through the use of checkpoint inhibitors.

The inventors have focused on developing an effective antigen delivery system. Despite more than 20 years of preclinical investigations on DC-based vaccines and many clinical trials in this field, designing an optimal effective DC-based vaccine for tumor rejection remains unclear. In preclinical modeling, some of the most effective approaches involve the administration of APCs that have been loaded with tumor-specific antigens. Various cells have been used, including splenocytes, macrophages, B cells, and dendritic cells. Using the Meth-A tumor model, the inventors showed that combination of CD11c+ MHCII^{lo} CD11b^{hi}, CD24^{lo}, CD40−, CD86^{lo} GM-CSF derived dendritic cells, or CD11c+ MHCII^{lo/int} CD11b^{hi}, CD24^{-/lo}, CD40^{-/lo}, CD86^{-lo} GM-CSF derived dendritic cells, with a proper neoepitope and optionally anti-CTLA4 were optimal tumor vaccines.

A tumor rejection assay was used to assess the efficacy of splenocytes, macrophages, BMDCs and DCs derived from monocytes. It was useful to employ monocyte-derived DCs, as this approach can be duplicated clinically with relative ease, and is thus translatable. Of the cells tested, GM-CSF-induced BMDCs were clearly superior to other APCs in mediating tumor regression, providing faster tumor clearance than did FLT-3-induced BMDCs. In this model, checkpoint inhibition with an anti-CTLA-4 antibody was useful for optimal tumor response for the Neo1 antigen.

It is perhaps not surprising that BMDCs mediated the best tumor regression, as they are known to be professional APCs that can mediate T cell expansion and antigen recognition. The first surprise in this study was the observation that immunization with peptide-loaded BMDCs acts both as antigen reservoir and professional antigen presenter. It is well-known that for DCs to mediate T cell maturation and clonal expansion, presumably prerequisites for tumor rejection in this model, the DCs must express “self” MHC from the perspective of the naive T cells. The C57Bl/6 BMDCs mediated effective tumor regression, though less rapidly than did the isogenic BALB/cJ BMDCs. Without being held to theory, one possible reason for this difference would be that the host mice would be expected to recognize and kill the C57Bl/6 BMDCs through alloreactivity, leading to a reduced persistence of the BMDCs, and thus a lower response. Another possibility would be that BMDCs with self MHC serve both as a reservoir for antigen as well as functioning as conventional APCs. In order to distinguish between these possibilities two more experiments were performed. The data from the β-2 microglobulin^{-/-} mice certainly suggests that BMDCs can function as peptide antigen reservoirs, facilitating cross-presentation by endogenous APCS, leading to tumor rejection. The data from OTI assay suggest that BMDCs can act as antigen presenters. It is shown herein that BMDCs can act as both antigen presenters and antigen reservoir.

Tumor rejection in this model is dependent on both CD4 and CD8 T cells, but the role of CD4 T cells was particularly strong. In fact, in control mice with CD4 T cells deleted, the Meth-A tumors grew faster than they did in control animals, suggesting that CD4 T cells help to mediate tumor control. In this model, it is clear that efficient tumor rejection depends on both CD4 and CD8 T cells, and effective immunization strategies should consider means of augmenting both responses.

Given the ability of the GM-CSF-induced BMDCs to mediate CD4 and CD8 reactivity in this model, it is surprising that DC-based clinical vaccine trials for cancer have not seen greater success. Without being held to theory, it is believed that the heterogeneity of DCs may offer some explanation. GM-CSF-induced BMDCs are a heterogeneous population of cells, based on their expression of CD11c, CD11b, MCHII, and costimulatory molecules. It was found herein that the optimal BMDCs in this model for mediating tumor regression were CD11c⁺ MHCII^{lo} CD11b^{hi}, CD24^{lo}, CD40⁻, CD86^{lo} GM-CSF derived dendritic cells. In an additional embodiment, it was found that the optimal BMDCs in this model for mediating tumor regression were CD11c⁺ MHCII^{lo/int} CD11b^{hi}, CD24^{-/lo}, CD40^{-/lo}, CD86^{-/lo} GM-CSF derived dendritic cells. This is not the expected phenotype for a DC that would mediate T cell responses, and so would be overlooked in most clinical studies of DCs. The population with the more conventional phenotype, CD11c⁺, MCHII^{hi}, CD11b⁺, and positive for CD86, CD40, and CD24, also mediated tumor regression in nearly all animals, though over a longer time course. This observation suggests that conventional DCs are less important than this novel population with lower MHC class II expression and absent costimulatory molecules. The mechanism of action of this population of cells merits further investigation.

In summary, GM-CSF-induced BMDCs mediate neo-epitope-specific tumor rejection, by functioning as both antigen reservoirs and presenters. The reservoir function which we believe is the major function does not require MHC restriction, as allo-reactive BMDCs were also able to mediate tumor regression. Both CD4 and CD8 T cells were required for tumor rejection, and the impact of CD4 T cells was particularly important. The phenotype of the BMDCs that best mediated tumor rejection was CD11c⁺ MHCII^{lo} CD11b^{hi}, CD24^{lo}, CD40⁻, CD86^{lo} GM-CSF derived dendritic cells. In another embodiment, the phenotype of the BMDCs that best mediated tumor rejection was CD11c⁺ MHCII^{lo/int} CD11b^{hi}, CD24^{-/lo}, CD40^{-/lo}, CD86^{-/lo} GM-CSF derived dendritic cells.

As used herein, dendritic cells (DCs) are antigen-presenting cells of the immune system. They engulf and process bits of bacteria, viruses, and other pathogens before presenting the relevant protein chain targets (antigenic peptides), to Cytotoxic T Lymphocytes (CTL), which recognize and kill virus-infected or cancer cells, and B-lymphocytes, which make antibodies. DCs also engulf cells which are damaged or dead, and are required to induce either a Type 1 response (activation), a Type 2 response (tolerant), or a Type 0 response (neutral). Because the same 20 amino acids make up body parts (self), as well as pathogens (non-self), DCs must evaluate not only the antigen structure, but also the cytokine and other signaling environment present at the time. This multi-layered system is in place to prevent auto-immunity, where the immune system mistakes self for non-self, as well as allergic responses, where a neutral response is required to maintain balance. This complex system of internal checks and balances is exploited by tumor cells, which arise from “self” cells. DCs have the capability of programming CD4⁺ and CD8⁺ CTL to recognize the MHC (self-protein ID complex) and associated peptide presented. However, the CTL must then decide whether to ignore the cell as self, or initiate lysis. The decision will often rely on the activation state of the CTL, the cytokine environment, and the presence or absence of cell-damage factors, e.g., heat-shock proteins, Toll-like receptor activation signals, and the like. During an active pathogen infection, these systems become activated and help steer the CTL response to a Type 1 attack mode.

In an aspect, provided herein are isolated CD11c⁺ MHCII^{lo} CD11^{hi}, CD24^{lo}, CD40⁻, CD86^{lo} cells, e.g., blood or bone marrow derived dendritic cells. Also provided is a composition comprising isolated CD11c⁺ MHCII^{lo} CD11b^{hi}, CD24^{lo}, CD40⁻, CD86^{lo} cells and a pharmaceutically acceptable excipient. In an embodiment, provided herein are isolated CD11c⁺ MHCII^{lo/int} CD11b^{hi}, CD24^{-/lo}, CD40^{-/lo}, CD86^{-/lo} dendritic cells, e.g., blood or bone marrow derived dendritic cells (e.g., isolated from a human subject). Also provided is a composition comprising isolated CD11⁺ MHCII^{lo/int} CD11b^{hi}, CD24^{-/lo}, CD40^{-/lo}, CD86^{-/lo} dendritic cells and a pharmaceutically acceptable excipient. In an embodiment, provided herein are isolated CD11c⁺ MHCII^{int} CD11b^{hi}, CD24^{-/lo}, CD40^{-/lo}, CD86^{-/lo} dendritic cells, e.g., blood or bone marrow derived dendritic cells (e.g., isolated from a human subject) and compositions comprising the dendritic cells and a pharmaceutically acceptable excipient. In another embodiment, provided herein are isolated CD11c⁺ MHCII^{lo/int} CD11b^{hi}, CD24^{lo}, CD40⁻, CD86^{lo} dendritic cells, e.g., blood or bone marrow derived dendritic cells (e.g., isolated from a human subject), and compositions comprising the dendritic cells and a pharmaceutically acceptable excipient. The terms “lo” (which signifies low level of marker expression), “int” (which signifies intermediate level of marker expression) and “hi” (which signifies high level of marker expression) as used herein are understood to refer to the levels of marker expression as commonly used in the field of immunology and are terms well known in the art. In an embodiment, the marker expression in the cells described herein is “lo,” “intermediate,” or “hi” relative to the same marker expression in mature dendritic cells (for example, mature human bone marrow-derived dendritic cells). In an embodiment, the marker expression in the cells described herein is “lo,” “intermediate,” or “hi” relative to the same marker expression in mature GM-CSF-derived dendritic cells (for example, mature human GM-CSF-derived dendritic cells). The marker expression can be assessed by assessing mRNA or protein expression using techniques known in the art.

In an embodiment, the cells are human cells, in which an MHCII protein is an HLA. In a particular embodiment, the HLA is HLA DR (Human Leukocyte Antigen-antigen D Related).

In an embodiment, the cells are GM-CSF-BMDC cells (e.g., human GM-CSF-BMDC). In certain embodiments, the cells are GM-CSF-BMDCs, FLT3L-BMDCs or Mo-DCs (e.g., human GM-CSF-BMDCs, FLT3L-BMDCs or Mo-DCs). In the case of GM-CSF-BMDCs and FLT3L-BMDCs, these cells can be obtained by a bone marrow biopsy of a subject. In an embodiment, the cells are human Mo-DCs (e.g., obtained from a blood sample of a subject).

In an aspect, a composition comprises an isolated CD11c⁺ MHCII^{lo} CD11b^{hi}, CD24^{lo}, CD40⁻, CD86^{lo} cells, e.g., blood or bone marrow derived dendritic cell. In an embodiment, a composition comprises an isolated CD11c⁺ MHCII^{lo/int} CD11b^{hi}, CD24^{-/lo}, CD40^{-/lo}, CD86^{-/lo} cells, e.g., blood or bone marrow derived dendritic cell. As used herein, the term “derived” means that the dendritic cells are prepared by differentiating monocytes or bone marrow cells.

In an embodiment, for any of the isolated populations of cells referred to in this disclosure, the isolated population is enriched in the dendritic cells expressing the specified biomarkers. In another embodiment, for any of the isolated populations of cells referred to in this disclosure, the dendritic cells expressing the specified biomarkers in the isolated population are substantially purified.

In an aspect, isolated CD11c⁺ MHCII^{lo} CD11b^{hi}, CD24^{lo}, CD40⁻, CD86^{lo} dendritic cells are prepared by

isolating monocytes from a cancer patient's peripheral blood mononuclear cells,

differentiating the monocytes to dendritic cells, using, for example GM-CSF, IL-4, or both GM-CSF and IL-4,

FACS sorting the differentiated cells based on MHCII and CD11c expression, and

isolating from the sorted cells a population of cells with the same phenotype as a mouse CD11c⁺ MHCII^{lo} CD11b^{hi}, CD24^{lo}, CD40⁻, CD86^{lo} GM-CSF derived dendritic cell.

In an aspect, isolated CD11c⁺ MHCII^{lo/int} CD11b^{hi}, CD24^{-/lo}, CD40^{-/lo}, CD86^{-/lo} dendritic cells are prepared by

isolating monocytes from a subject's peripheral blood mononuclear cells,

differentiating the monocytes to dendritic cells, using, for example GM-CSF, IL-4, or both GM-CSF and IL-4,

FACS sorting the differentiated cells based on MHCII and CD11c expression, and

isolating from the sorted cells a population of CD11c⁺ MHCII^{lo} CD11b^{hi}, CD24^{-/lo}, CD40^{-/lo}, CD86^{-/lo} dendritic cells.

In another aspect, isolated CD11c⁺ MHCII^{lo} CD11b^{hi}, CD24^{lo}, CD40⁻, CD86^{lo} dendritic cells are prepared by

obtaining bone marrow cells from a cancer patient,

differentiating the bone marrow cells to dendritic cells using, for example, GM-CSF,

FACS sorting the differentiated cells based on MHCII and D11c expression,

isolating from the sorted cells a population of cells with the same phenotype as a mouse CD11c⁺ MHCII^{lo} CD11b^{hi}, CD24^{lo}, CD40⁻, CD86^{lo} GM-CSF derived dendritic cell.

In another aspect, isolated CD11c⁺ MHCII^{lo/int} CD11b^{hi}, CD24^{-/lo}, CD40^{-/lo}, CD86^{-/lo} dendritic cells are prepared by

obtaining bone marrow cells from a subject,

differentiating the bone marrow cells to dendritic cells using, for example, GM-CSF,

FACS sorting the differentiated cells based on MHCII and D11c expression,

isolating from the sorted cells a population of CD11c⁺ MHCII^{lo/int} CD11b^{hi}, CD24^{-/lo}, CD40^{-/lo}, CD86^{-/lo} dendritic cells.

The subject can be a cancer patient, or a healthy subject.

The population of cells with the same phenotype as a mouse CD11c⁺ MHCII^{lo} CD11b^{hi}, CD24^{lo}, CD40⁻, CD86^{lo} GM-CSF derived dendritic cell or a mouse CD11c⁺ MHCII^{lo/int} CD11b^{hi}, CD24^{lo}, CD40⁻, CD86^{lo} GM-CSF derived dendritic cell can be identified by RNA sequencing of different types of dendritic cells, e.g. human dendritic cells, which be compared with the RNA expression pattern of “CD11c⁺ MHCII^{lo/int} CD11b^{hi}, CD24^{lo}, CD40⁻, CD86^{lo} GM-CSF derived dendritic cell”. The most similar subpopulation of human dendritic cells to the above mouse population may be used in combination with neoepitopes as a cancer vaccine in human. In certain embodiments, the human dendritic cells are isolated based on the expression of at least 1, 2, 3, 4, 5, 6, 7, 8, 9 or 10 or more (e.g., at least 3, or at least 5, or at least 7) of the markers listed in any of Tables 1-4 (e.g., Tables 2-4, Table 1, Table 2, Table 3, or Table 4) as shown for P6 cells (immature dendritic cells), optionally, in addition to the expression of CD11c⁺ MHCII^{lo/int} CD11b^{hi} or in addition to the expression of CD11c⁺ MHCII^{lo/int} CD11b^{hi} CD24^{-/lo}, CD40^{-/lo}, CD86^{-/lo}. For example, where a marker listed in any of Tables 1-4 is shown to be expressed at an increased level in immature dendritic cells relative to mature dendritic cells, the immature dendritic cells of interest can be isolated based on such increased expression of the marker. In another example, where a marker listed in any of Tables 1-4 is shown to be expressed at a decreased level in immature dendritic cells relative to mature dendritic cells, the immature dendritic cells of interest can be isolated based on such decreased expression of the marker. The foregoing applies to any marker, any combination of markers, or all markers listed in Tables 1-4. The described human dendritic cells can be isolated from blood or bone marrow of a human patient (e.g., for use in therapeutic compositions described herein). In an embodiment, the isolating of cells comprises one or more steps of FACS sorting based on the expression of 1, 2, 3, 4, 5, 6, 7, 8, 9, 10, or more, marker(s) described herein.

The terms “marker” and “biomarker” are used interchangeably in this disclosure.

In an embodiment, provided herein are isolated dendritic cells having at least 1, 2, 3, 4, 5, 6, 7, 8, 9 or 10 (e.g., at least 3 or at least 5) of the following biomarkers: CD91^{hi}, LOX1^{hi}, TLR2^{hi}, CD80^{-/lo}, CD36^{lo}, CD209a^{-/lo}, mannose receptor C-type 1^{hi}, macrophage scavenger receptor 1^{hi}, TLR1^{hi}, TLR6^{hi}, and TLR7^{hi}, optionally, in addition to CD11c⁺ MHCII^{lo/int} CD11b^{hi}, or in addition to CD11c⁺ MHCII^{lo/int} CD11b^{hi}, CD24^{-/lo}, CD40^{-/lo}, CD86^{-/lo}In an embodiment, provided herein are isolated dendritic cells having at least 3, 4, 5, 6, 7, 8, 9 or 10 (e.g., at least 3, at least 4, at least 5, at least 6, or at least 7) of the following biomarkers: CD91^{hi}, LOX1^{hi}, TLR2^{hi}, CD80^{-/lo}, CD36^{lo}, CD209a^{-/lo}, mannose receptor C-type 1^{hi}, macrophage scavenger receptor 1^{hi}, TLR1^{hi}, TLR6^{hi}, TLR7^{hi}, CD11c⁺ MHCII^{lo/int} CD11b^{hi}, CD24^{-/lo}, CD40^{-/lo}, and CD86^{-/lo}. The described dendritic cells can be isolated from blood or bone marrow of a human patient (e.g., for use in therapeutic compositions described herein).

In an embodiment, provided herein are isolated dendritic cells comprising any one or more (e.g., at least 1, 2, 3, 4, 5, or 6) of the following biomarkers: C1s1^{hi}, Cfb^{hi}, Fos^{hi}, Hp^{hi}, Il18^{hi}, Il1a^{hi}, Il1f9^{hi}, Serpine1^{hi} and Serpinf1^{lo}, optionally in addition to CD11c⁺ MHCII^{lo/int} CD11b^{hi}, CD24^{-/lo}, CD40^{-/lo}, CD86^{-/lo} In another embodiment, provided herein are isolated dendritic cells comprising one or more of the biomarkers Fos^{hi}, Il18^{hi}, Il1a^{hi}, Il1f9^{hi}, Il1rl1^{lo} and or Tlr9^{lo}, optionally in addition to CD11c⁺ MHCII^{lo/int} CD11b^{hi}, CD24^{-/lo}, CD40^{-/lo}, CD86^{-/lo}. In another embodiment, provided herein are isolated dendritic cells comprising any one or more (e.g., at least 1, 2, 3, 4, 5, 6, 7, 8, 9, or 10) of the following biomarkers: Ccr7^{lo}, Cd40^{lo}, Cd83^{lo}, Fgfr1^{lo}, Fscn1^{lo}, H2-DMb2^{lo}, H2-Oa^{lo}, H2-Ob^{lo}, H2-Aa^{lo}, H2-Ab1^{lo}, H2-Ea-ps^{lo}, H2-Eb1^{lo}, Il18^{hi}, Il1a^{hi}, Il1f9^{hi}, Jak2^{lo}, Stat4^{lo}, and Tlr9^{lo}, optionally in addition to CD11c⁺ MHCII^{lo/int} CD11b^{hi}, CD24^{-/lo}, CD40^{-/lo}, CD86^{-/lo}. In another embodiment, provided herein are isolated dendritic cells comprising any one or more (e.g., at least 1, 2, 3, 4, 5, or 6) of the following biomarkers Fgfr1^{lo}, Fyn^{lo}, Itgae^{lo}, Mylk^{lo}, Ptk2^{lo}, Tln2^{hi}, Tlr9^{lo}, and Tspan2^{lo}. In another embodiment, provided herein are isolated dendritic cells comprising any one or more (e.g., at least 1, 2, 3, 4, 5, or 6) of the following biomarkers: Fgfr1^{lo}, Fos^{hi}, Fyn^{lo}, Ppp1r14a^{lo}, Ptk2^{lo}, Tln2^{hi}, and Tlr9^{lo}, optionally in addition to CD11c⁺ MHCII^{lo/int} CD11b^{hi}, CD24^{-/lo}, CD40^{-/lo}, CD86^{-/lo}. The described dendritic cells can be isolated from blood or bone marrow of a human patient (e.g., for use in therapeutic compositions described herein).

Once the CD11c⁺ MHCII^{lo} CD11b^{hi}, CD24^{lo}, CD40⁻, CD86^{lo} dendritic cells have been isolated, they can be pulsed with a patient tumor neoepitope peptide or a nucleic acid encoding a neoepitope peptide of a cancer patient to provide a dendritic cell-neoepitope composition. In another embodiment, once the CD11c⁺ MHCII^{lo/int} CD11b^{hi}, CD24^{-/lo}, CD40^{-/lo}, CD86^{-/lo} dendritic cells have been isolated, they can be pulsed with a tumor neoepitope peptide or a nucleic acid encoding a neoepitope peptide of a cancer patient to provide a dendritic cell-neoepitope composition. In certain embodiments, the cells can be pulsed with a population of tumor neoepitope peptides or nucleic acids encoding neoepitope peptides, e.g., two or more peptides or nucleic acids, three or more peptides ore nucleic acids, four or more peptides or nucleic acids, or five or more peptides or nucleic acids, or populations with even larger numbers of peptides or nucleic acids, as desired.

Neoepitope peptides can be synthesized prior to combining them with the dendritic cells, or prior to pulsing the dendritic cells with the peptides. Neoepitope peptides can be chemically synthesized using well known methods of peptide synthesis. Alternatively, neoepitope peptides can be expressed recombinantly using well known molecular biology methods.

Neoepitopes include neoepitopes known in the art as well as neoepitopes selected by the methods described in US2015/0252427 and WO2016/040110, incorporated by reference herein for their disclosure of neoepitopes and methods of identifying neoepitopes in a cancer patient. In a specific aspect, the neoepitopes are specific to a tumor from the cancer patient from whom the dendritic cells have been isolated. In another specific aspect, the neoepitopes are specific to a tumor of the cancer patient. In an aspect, the neoepitopes are not from known cancer-causing pathways. In another aspect, the neoepitopes are somatic or passenger mutations from the patient's tumor. In an aspect, the conformational stability of the neoepitope bound to an MHCI or MHCII protein as determined by molecular modeling or experiment is higher compared to the corresponding wild type epitope.

Specifically, in WO2014/052707, incorporated herein by reference in its entirety and particularly for its teaching of the determination of tumor-specific epitopes, a novel index called the Differential Agretopic Index (DAI) was described. The DAI is an improvement over algorithms such as NetMHC in the selection of tumor-specific epitopes. The selection of tumor-specific epitopes can be further improved by relying (e.g., in addition to DAI) on the conformational stability of the peptides when the peptide is bound to an MHC protein, which is a strong predictor of immunological outcome. Specifically, WO2016/040110 is incorporated herein by reference in its entirety, and particularly for its teaching of using the conformational stability of peptides in identification and selection of tumor-specific epitopes. The immunogenic neo-epitopes were unexpectedly found to have higher conformational stability than the corresponding wild type sequence. That is, the mutations that result in higher conformational stability of the peptide relative to the wild type peptide are more likely to be immunogenic.

In an aspect, the DAI score (the numerical difference between the NetMHC scores of the mutated epitope and its un-mutated counterpart) allows significant enrichment for the extremely small number of truly immuno-protective neo-epitopes from among the hundreds of putative neo-epitopes identified by the NetMHC algorithm. Peptide conformational stability, expressed as the fluctuations observed during molecular dynamics simulations, but also determinable via other computational and experimental techniques, is another tool that suggests a novel correlate with immunogenicity. The majority of the neo-epitopes with high DAI rankings are predicted to interact with the MHC in a more stable fashion than their wild-type counterparts; in these cases, alteration of the anchor residues yields a more rigidly bound peptide. Of course, methods other than the DAI can be used to determine putative neo-epitope sets, and other methods than fluctuations observed during molecular dynamics simulations can be used to assess the conformational stability of the neo-epitopes.

In an embodiment, a method of identifying immunologically protective neo-epitopes in a cancer patient comprises

providing a putative neo-epitope set,

determining the conformational stability of at least a portion of each putative neo-epitope in the putative neo-epitope set bound to an MHC I or MHC II protein,

selecting from the putative neo-epitope set the immunologically protective neo-epitopes, wherein the immunologically protective neo-epitopes have higher conformational stability compared to the corresponding wild type epitopes when bound to the MHC I or MHC II protein,

optionally producing a pharmaceutical composition comprising a pharmaceutically acceptable carrier and one or more immunologically protective neo-epitope peptides, one or more polypeptides containing the immunologically protective neo-epitopes, or one or more polynucleotides encoding the one or more immunologically protective neo-epitopes, and

optionally administering the pharmaceutical composition to the cancer patient.

The putative neo-epitope set can be identified using the DAI as described herein, or can be determined using the NetMHC scores, a peptide-MHC protein on-rate, a peptide-MHC protein off-rate, peptide solubility and/or other physical and/or chemical properties of the peptides. In an embodiment, the neoepitope peptide(s) identified using DAI and/or the conformational stability of the peptide(s) (i.e., conformational stability of at least a portion of the peptide(s) bound to an MHC I or MHC II protein) are used to pulse the dendritic cells.

For a peptide in a class I or class II MHC binding groove, the conformation is the structure the peptide adopts within the groove, as commonly although not exclusively determined via X-ray crystallography or examined by computational modeling (see, for example pmid 17719062). Stability is defined as the extent to which the conformation fluctuates (or moves) around this conformation, which can be measured or estimated using thermodynamic, spectroscopic, computational, crystallographic, or hydrogen exchange techniques. Stability can also include entropy as well as other dynamic processes. Thermodynamic techniques include, but are not limited to, measurements of peptide binding entropy changes by calorimetry, van′t Hoff analyses, or Eyring analyses (see, for example, pmid 12718537). Spectroscopic techniques include, but are not limited to, examination of peptide motion by nuclear magnetic resonance, fluorescence, or infra-red spectroscopy (see, for example, pmid 19772349). Computational techniques include, but are not limited to, molecular dynamics simulations or Monte Carlo sampling (see, for example, pmid 21937447). Crystallographic techniques include, but are not limited to, comparison of multiple X-ray structures of the same peptide-MHC complex, examination of electron density, examination of crystallographic temperature factors, or examination of alternate peptide conformations present in one X-ray structure (see, for example, pmid 17719062). Hydrogen exchange techniques include, but are not limited to, measurements of the rates of hydrogen exchange or the extent of exchange at a given time point by NMR or mass spectrometry. The techniques described above are techniques that can be used to determine the conformational stability of peptides (i.e., conformational stability of at least a portion of peptides bound to an MHC I or MHC II protein).

As used herein, the term conformational fluctuations refers to either amplitude or frequency of motion around a structure. Therefore, with higher conformational stability, an epitope has fewer fluctuations around a structure. An equivalent way of describing conformational fluctuations is that with higher conformational stability, there is less motion of the peptide. The term fluctuations can be used interchangeably with motion, entropy or other terms that describe dynamic motion in a peptide.

Examples of meaningful reductions in conformational stability are:
For thermodynamic measurements, reduction of the entropy of peptide binding (ΔS°) by 3 calK/mol or more.
For crystallographic analyses, elimination of alternate conformations in a refined structure, elimination of electron density gaps in a 2Fₒ-F_{c} electron density map, or reductions of temperature factors for atoms of the peptide by 10% or more.
For measurements using nuclear magnetic resonance, increases in order parameters for atoms of the peptide by 10% or more.
For measurements using fluorescence anisotropy, increases in steady state anisotropy values for a fluorescently labeled or intrinsically fluorescent peptide of 20% or more or decreases in correlation times of 20% or more.
For computational analyses, decreases in the root mean square fluctuations of atoms of the peptide by 0.5 Å or more.
For analyses of hydrogen exchange by NMR or mass spectrometry, decreases in the rates of hydrogen exchange at individual amides or of amino acid fragments of 15% or more, or decreases in the extent of exchange at a particular time point of 15% or more.

Any one of the above measures of higher conformational stability can be used to determine that a mutant peptide has a higher conformational stability than the wild type peptide. The quantification of higher conformational stability for a neo-epitope compared to the wild-type sequence is thus dependent upon the technique used to determine the conformational stability. However, such techniques are well-known in the art and one of ordinary skill in the art could readily determine if the mutant epitope has higher conformational stability then a wild-type epitope using a specified technique.

Conformational stability may be determined for the entire epitope or specific regions (e.g., the peptide center, N- or C-terminus, etc.).

In a specific embodiment, once the putative neo-epitope set has been selected, the root mean squared fluctuations (RMSF) of at least a portion of each epitope in the putative neo-epitope set bound to an MHC I or MHC II protein are determined as a measure of the conformational stability of the peptides. The root mean squared fluctuations are determined for the C-terminal portion of the peptide, the central portion of the peptide, the N-terminal portion of the peptide, or the entire peptide. It was unexpectedly found that mutant peptides which fail to elicit an immunological response have a high instability, particularly C-terminal instability. In the studies presented herein, the average C-terminal RMSF was 0.9 Å, and peptides with a C-terminal RMSF below this value were immunogenic. In specific embodiments, it is preferred that root mean squared fluctuations of at least a portion of the α-carbons of each epitope in the putative neo-epitope set bound to an MHC I or MHC II protein is less than 2 Å, less than 1.5 Å less than 1.2 Å, or less than 0.9 Å. Thus, C-terminal stability is a predictor of immunogenicity. Immunologically protective neo-epitopes are selected from the putative epitope set as epitopes having a root mean squared fluctuation of less than 2 Å, less than 1.5 Å less than 1.2 Å, or less than 0.9 Å.

In an aspect, the MHC protein is an MHC I protein and the immune response is a CD8+ response. Exemplary MHC I proteins include the mouse H-2k^{d}, H-2k^{b} and H-2D^{d} peptides and the human HLA protein, such as HLA-A, HLA-B and HLA-C, specifically HLA-A*0201. Thus, in an aspect, the method further comprises assaying the CD8 T-cell response of the neo-epitopes.

In another aspect, the MHC peptide is an MHC II protein and the response is a CD4+ response. Exemplary MHC II proteins include HLA-DR, HLA-DP, HLA-DQ. Thus, in an aspect the method further comprises assaying the CD4 T-cell response of the neo-epitopes.

In yet another aspect, the immunologically protective neo-epitopes have a measured IC50 for H-2K^{d} or HLA of greater than 100 nM or greater than 500 nM.

In an embodiment, the putative neo-epitope set is determined using the DAI determined by the following method:

sequencing at least a portion of the cancer patient's RNA or DNA in both a healthy tissue and a cancer tissue, to produce a healthy tissue RNA or DNA sequence and a cancer tissue RNA or DNA sequence,

comparing the healthy tissue RNA or DNA sequence and the cancer tissue RNA or DNA sequence and identifying differences between the healthy tissue RNA or DNA sequence and the cancer tissue RNA or DNA sequence to produce a difference DNA marker set,

analyzing the difference DNA marker set to produce a tumor-specific epitope set, wherein the tumor-specific epitope set comprises one or more tumor-specific epitopes,

providing a numerical score called the Differential Agretopic Index for each epitope in the tumor-specific epitope set, wherein the Differential Agretopic Index is calculated by subtracting a score for a normal epitope from a score for the tumor-specific epitope, and

ranking the tumor-specific epitope set according to the Differential Agretopic Index and selecting a putative neo-epitope set from the tumor-specific epitope set based on the ranking.

The method further comprises providing a numerical score for each epitope in the tumor-specific epitope set or the MHC-restricted tumor-specific epitope set, wherein the numerical score is calculated by subtracting a score for the normal epitope (non-mutated) from a score for the tumor-specific epitope (mutated). The numerical score for the normal epitope is subtracted from the numerical score for the mutant cancer epitope, and a numerical value for the difference is obtained—the Differential Agretopic Index (DAI) for the epitope. The putative epitopes can be ranked on basis of the DAI. In this ranking, broadly speaking, the higher the difference for a given epitope, the higher the probability that immunization with it shall be protective against the tumor. In a specific embodiment, the highest ranked epitopes are used to immunize an individual. Further, the method can comprise ranking the tumor specific-epitope set or the MHC-restricted tumor-specific epitope set by the Differential Agretopic Index for each epitope in the set. In an aspect, the method further comprises using the ranking by Differential Agretopic Index (DAI) to identify a subset of 10 to 50 top-ranked tumor specific-epitopes. Top-ranked means the epitopes with the most favorable DAI.

In a specific embodiment, analyzing the difference DNA marker set to produce a tumor-specific epitope set is independent of whether one or more tumor-specific epitopes are related to cancer-causing pathways. Prior methods for analyzing the DNA of cancer patients focused on the genetic mechanisms that cause cancer or that drive cancer, while the present approach is agnostic about that issue. The approach described herein is aimed to attack cancer at any point where it is different from the normal, regardless of whether that difference is responsible for causing cancer or not. A major consequence of this difference is that the other approaches rely mostly on deciding which existing (or future) medicines to use for each patient, and not on designing a medicine for each patient. The present method focuses on designing a medicine to treat a particular tumor.

An “isolated” or “purified” peptide is substantially free of cellular material or other contaminating polypeptide from the cell or tissue source from which the protein is derived, or substantially free of chemical precursors or other chemicals when chemically synthesized. The language “substantially free of cellular material” includes preparations of polypeptide in which the polypeptide is separated from cellular components of the cells from which it is isolated or recombinantly produced. Immunologically protective neo-epitope peptides generally have lengths of 7 to 25 amino acids, specifically 8 to 15 amino acids, and more specifically 8 to 10 amino acids.

In an embodiment, a separate peptide corresponding to each immunologically protective neoepitope is employed. In another embodiment, a polypeptide containing two or more immunologically protective neoepitopes is employed. One polypeptide containing multiple immunologically protective neoepitopes optionally separated by non-epitope linkers can be employed. Such polypeptides can be readily designed by one of ordinary skill in the art.

In certain embodiments, instead of immunologically protective neoepitope peptides, a pharmaceutical composition comprises one or more polynucleotides encoding the peptides. The peptides can all be expressed from the same polynucleotide molecule, or from multiple polynucleotide molecules.

In an aspect, the neoepitope peptides contain at least one substitution modification relative to the neoepitope or one or more nucleotides at the 5′3 or 3′ end of the peptide that is not found in the neoepitope. In another aspect, a detectable label is attached to the neoepitope.

“Polynucleotide” or “nucleic acid sequence” refers to a polymeric form of nucleotides at least 5 bases in length. The nucleotides can be ribonucleotides, deoxyribonucleotides, or modified forms of either nucleotide. Polynucleotides can be inserted into a recombinant expression vector or vectors. The term “recombinant expression vector” refers to a plasmid, virus, or other means known in the art that has been manipulated by insertion or incorporation of the peptide genetic sequence. The term “plasmids” generally is designated herein by a lower case “p” preceded and/or followed by capital letters and/or numbers, in accordance with standard naming conventions that are familiar to those of skill in the art. Plasmids disclosed herein are either commercially available, publicly available on an unrestricted basis, or can be constructed from available plasmids by routine application of well-known, published procedures. Many plasmids and other cloning and expression vectors are well known and readily available, or those of ordinary skill in the art may readily construct any number of other plasmids suitable for use. These vectors may be transformed into a suitable host cell to form a host cell vector system for the production of a polypeptide.

The peptide-encoding polynucleotides can be inserted into a vector adapted for expression in a bacterial, yeast, insect, amphibian, or mammalian cell that further comprises the regulatory elements necessary for expression of the nucleic acid molecule in the bacterial, yeast, insect, amphibian, or mammalian cell operatively linked to the nucleic acid molecule encoding the peptides. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (i.e., ATG) in front of a protein-encoding gene, splicing signals for introns (if introns are present), maintenance of the correct reading frame of that gene to permit proper translation of the mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter. By “promoter” is meant minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included.

A pharmaceutical composition (e.g., a vaccine) comprises an isolated CD11c⁺ MHCII^{lo} CD11b^{hi}, CD24^{lo}, CD40⁻, CD86^{lo} blood or bone marrow derived dendritic cell derived from the blood or bone marrow of a cancer patient and at least one isolated immunologically protective neoepitope peptide (or RNA or DNA encoding such epitope peptides) and a pharmaceutically acceptable carrier. In another embodiment, a pharmaceutical composition (e.g., a vaccine) comprises an isolated CD11c⁺ MHCII^{lo/int} CD11b^{hi}, CD24^{-/lo}, CD40^{-/lo}, CD86^{-/lo} blood or bone marrow derived dendritic cell derived from the blood or bone marrow of subject and at least one isolated immunologically protective neoepitope peptide (or RNA or DNA encoding such epitope peptides) of a cancer patient and a pharmaceutically acceptable carrier, wherein the subject is a healthy subject or the cancer patient. Pharmaceutically acceptable excipients include, for example, diluents, preservatives, solubilizers, emulsifiers, and adjuvants. As used herein “pharmaceutically acceptable excipients” are well known to those skilled in the art. In an embodiment, a pharmaceutical composition allows for local delivery of the active ingredient, e.g., delivery directly to the location of a tumor.

In specific embodiment, a pharmaceutical composition comprises 1 to 100 immunologically protective neo-epitope peptides, specifically 3 to 20 immunologically protective neo-epitope peptides. In another embodiment, a pharmaceutical composition comprises a polypeptide containing 1 to 100 immunologically protective neo-epitopes, specifically 3 to 20 immunologically protective neo-epitopes. In another aspect, a pharmaceutical composition comprises a polynucleotide encoding 1 to 100 immunologically protective neo-epitopes, specifically 3 to 20 tumor-specific immunologically protective neo-epitopes.

In an embodiment, pharmaceutical compositions suitable for intravenous, intramuscular, subcutaneous, intradermal, nasal, oral, rectal, vaginal, or intraperitoneal administration conveniently comprise sterile aqueous solutions of the active ingredient with solutions which are preferably isotonic with the blood of the recipient. Such formulations can be conveniently prepared by dissolving the peptide in water containing physiologically compatible substances, such as sodium chloride (e.g., 0.1-2.0 M), glycine, and the like, and having a buffered pH compatible with physiological conditions to produce an aqueous solution, and rendering said solution sterile. These can be present in unit or multi-dose containers, for example, sealed ampoules or vials.

Additional pharmaceutical methods can be employed to control the duration of action. Controlled release preparations can be achieved through the use of polymer to complex or absorb the peptides or nucleic acids. The controlled delivery can be exercised by selecting appropriate macromolecules (for example polyester, polyamino acids, polyvinyl, pyrrolidone, ethylenevinylacetate, methylcellulose, carboxymethylcellulose, or protamine sulfate) and the concentration of macromolecules as well as the methods of incorporation in order to control release. Another possible method to control the duration of action by controlled-release preparations is to incorporate a protein, peptides and analogs thereof into particles of a polymeric material, such as polyesters, polyamino acids, hydrogels, polylactic acid) or ethylene vinylacetate copolymers. Alternatively, instead of incorporating these agents into polymeric particles, it is possible to entrap these materials in microcapsules prepared, for example, by coacervation techniques or by interfacial polymerization, for example, hydroxy-methylcellulose or gelatin-microcapsules and poly(methylmethacylate) microcapsules, respectively, or in colloidal drug delivery systems, for example, liposomes, albumin microspheres, microemulsions, nanoparticles, and nanocapsules or in macroemulsions.

Local administration to the afflicted site can be accomplished through means known in the art, including, but not limited to, topical application, injection, and implantation of a porous device containing cells recombinantly expressing the peptides, implantation of a porous device in which the peptides are contained.

In an embodiment, the composition further comprises an immune-modulating agent. Exemplary immune-modulating agents include TLR ligands such, for example, CpG oligonucleotide DNA (a TLR9 ligand), lipopeptides and lipoproteins (TLR and TLR2 ligands), poly I:C and double stranded RNA (TLR3 ligands), lipopolysaccharide (TLR4 ligand), diacyl lipopeptide (TLR6 ligands), imiquimod (a TLR7 ligand), and combinations of TLR ligands. Another exemplary immune-modulating agent is an antibody such as anti-cytotoxic T-lymphocyte antigen-4 antibody (anti-CTLA-4), or an antibody blocking Programmed Death 1 (PD1) or a PD1 ligand.

The immunogenic composition optionally comprises an adjuvant. Adjuvants in general comprise substances that boost the immune response of the host in a non-specific manner. Selection of an adjuvant depends on the subject to be vaccinated. Preferably, a pharmaceutically acceptable adjuvant is used. For example, a vaccine for a human should avoid oil or hydrocarbon emulsion adjuvants, including complete and incomplete Freund's adjuvant. One example of an adjuvant suitable for use with humans is alum (alumina gel).

In an embodiment, provided herein is a method of preparing a therapeutic composition, comprising combining one or more neoepitope peptides with a cell as disclosed herein. In an embodiment, the cells are pulsed with the neoepitope peptides. In another embodiment, the neoepitope peptides are synthesized prior to combining with the cell (e.g., prior to pulsing the cell with the neoepitope peptides). In another embodiment, the neoepitope peptides are identified using Differential Agretopic Index (DAI). In another embodiment, the neoepitope peptides are identified by conformational stability (i.e., conformational stability of at least a portion of the peptide(s) bound to an MHC I or MHC II protein).

In an embodiment, an immunotherapy method comprises administering to a cancer patient a composition comprising an isolated CD11c⁺ MHCII^{lo} CD11b^{hi}, CD24^{lo}, CD40⁻, CD86^{lo} blood or bone marrow derived dendritic cell derived from the blood or bone marrow of the cancer patient, and a neoepitope peptide or nucleic acid molecule encoding the neoepitope peptide, wherein the neoepitope peptide is specific to a tumor from the cancer patient. In another embodiment, an immunotherapy method comprises administering to a cancer patient a composition comprising an isolated CD11c⁺ MHCII^{lo/int} CD11b^{hi}, CD24^{-/lo}, CD40^{-/lo}, CD86^{-/lo} blood or bone marrow derived dendritic cell derived from the blood or bone marrow of a subject such as the cancer patient or a healthy subject, and a neoepitope peptide or nucleic acid molecule encoding the neoepitope peptide, wherein the neoepitope peptide is specific to a tumor from the cancer patient. Immunotherapy, unlike cytotoxic drugs, radiation, and surgery, stimulates the immune system to recognize and kill tumor cells.

As used herein, a patient is a mammal, such as a mouse or a human, specifically a human patient.

The compositions and methods described herein are applicable to all cancers including solid tumor cancers, e.g., those of the breast, prostate, ovaries, lungs and brain, and liquid cancers such as leukemias and lymphomas.

The methods described herein can be further combined with additional cancer therapies such as radiation therapy, chemotherapy, surgery, and combinations thereof.

The invention is further illustrated by the following non-limiting examples.

### EXAMPLES

### Materials and Methods

Mice, Tumors and Peptides.

C57BL/6, BALB/cJJ and B6. 129P2-B2mtm1Unc mice (6 wks female) were purchased from the Jackson Laboratory. Mice were maintained in the virus free mouse facilities at the University of Connecticut Health Center and their use was approved and monitored by the Institutional Animal Care and Use Committee. MethA is a fibrosarcoma induced by methylcholanthrene in a female BALB/cJ mouse. MethA ascites were used for passage and challenge. Six million ascites cells were inoculated intraperitoneally in BALB/cJ mice for 4 days before each tumor challenge. B16-OVA melanoma cell line was generously donated by Dr. Nick Restiffo laboratory. Neo1 a neoepitope of the BALB/cJ MethA-fibrosarcoma, was synthesized by JPT Peptide Technologies GmbH. Long peptide SIINFEHL (SEQ ID NO: 2) was synthesized by Genemed company.

Immunization.

Splenocytes, bone marrow derived day 7 DCs or MQ and monocytes derived day 3 DCs were pulsed with 40 microgram/mouse Neo1 or LP SIINFEHL for 2 hours. Cells were washed 4 times before the ID injection.

Tumor Challenge.

BALB/cJ mice (6 wks) were immunized twice weekly with intradermal injection of either BMDCs, Splenocytes, Macrophages or Monocytes-derived DCs pulsed with either DMSO as control or Neo1. The mice were shaved 2 days before tumor challenge. Seven days after the last immunization, 95,000 live MethA ascites cells (viability>98% by trypan blue exclusion) were injected intradermally on the lower right flank of mice. Tumor diameters were measured by calipers twice a week. Mice were sacrificed when tumors ulcerated, reached a maximum diameter of 15 mm, or when mice showed any sign of discomfort.

BMDCs.

2-3 million bone marrow cells per ml of 6-8 wks old mice were cultured in complete RPMI supplemented with 20 ng per ml recombinant murine GM-CSF (Peprotech) and incubated at 37° C., in 5% CO₂ incubator for 7 days. Cells were fed at day 3 with the same amount of media.

BMDMs.

Bone marrow-derived macrophages (BMDMs) were isolated from 6 wks week old BALB/cJJ of mice (vendor) by flushing femurs and tibias with DMEM. Cells were then incubated overnight in a tissue culture-treated 25 cm²-flasks at 37° C. with 5% CO₂. The following day, 1×10⁷ suspension cells were maintained in 10-cm² bacteriological Petri dishes (BD-Falcon) for three days with DMEM supplemented with 10% FBS, 20% L292-cell conditioned media, 0.01% HEPES, 0.01% sodium pyruvate, and 0.01% L-glutamine. Cultures were supplemented with five ml of the above-described medium and seven days after isolation, cell monolayers were exposed to ice-cold PBS and were recovered by scraping to be used for immunization.

Monocyte Derived DCs.

Peripheral blood mononuclear cells of 8 wks old female BALB/cJJ mice were isolated using Lymphoprep™ and SepMate™ from StemCell Technologies Co. Monocytes were isolated from PBMCs using EasySep™ Mouse Monocyte Isolation Kit. 500K monocytes/ml were cultured in complete RPMI (RPMI-1640, 10% FBS, 1% Pen-Strep/L-glutamine, 1% none essential amino acids, 1% sodium pyruvate and 0.1% 2-b-mercaptoethanol) supplemented with 50 ng/ml GM-CSF (Peprotech) plus 25 ng/ml IL-4 (StemCell Technologies) in 24-well plates for 3 days.

In Vivo Tumor Response: Flow Cytometry.

The antibodies specific for FITC CD11c (clone N418), Fixable Viability Dye eFluor® 780 and PE NK1.1 (clone PK136) were purchased from eBioscience. The antibodies specific for PE CD4 (clone GK1.5), PE/Cy7 CD8a (Clone 53-6.7) and PerCP MHCII (clone M5/114,15.2) were purchased from Biolegend. V500 CD11b (clone M1/70), APC CD86 (clone GLl[RUO]), CD40 (clone 3/23), CD24 (clone M1/69) were purchased from BD Biosciences. Flow cytometry and cell sorting were performed using Miltenyi Biotec MACSQuant® analyzer and BD LSR II-B, respectively. Analysis was done using FlowJo® software.

Statistical Analysis:

P-values for group comparisons were calculated using a two-tailed nonparametric Mann-Whitney test, using GraphPad Prism® 5.0 (GraphPad).

### Example 1: Tumor Protection of BMDCs, BMDMs and Splenocytes

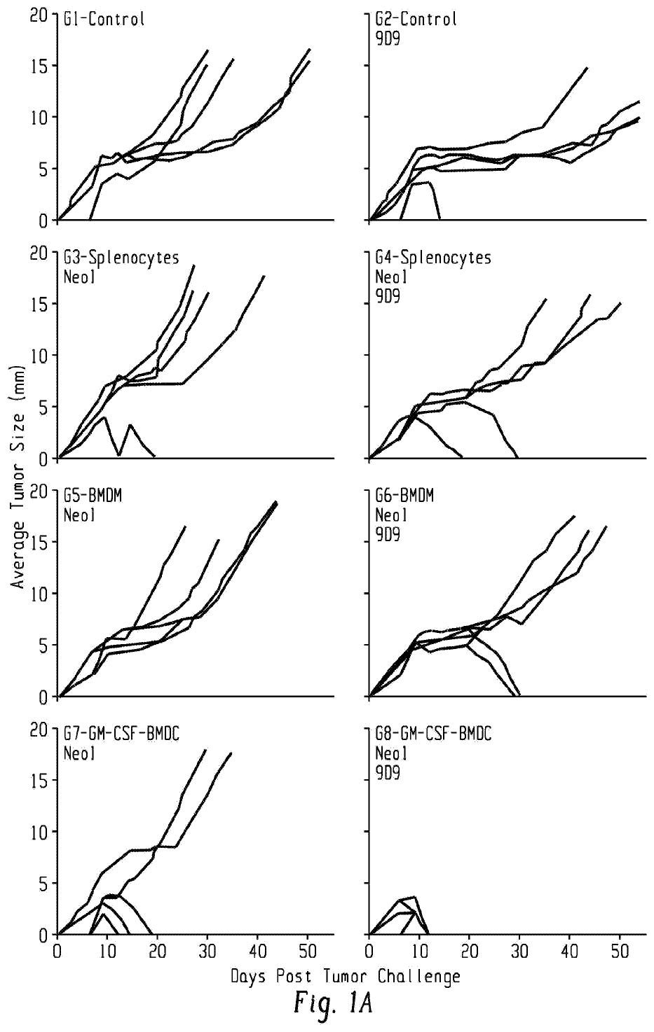

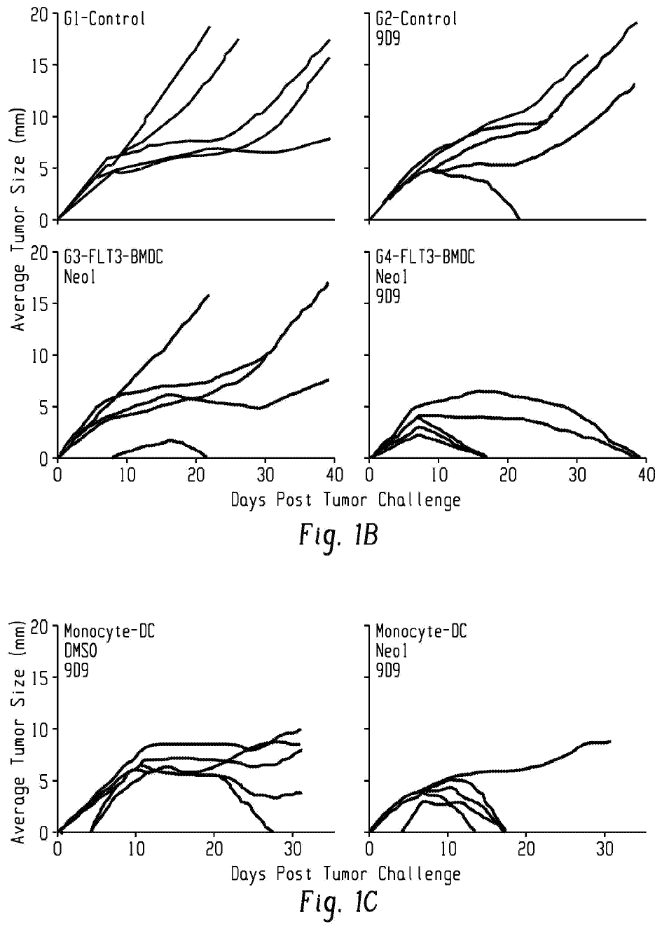

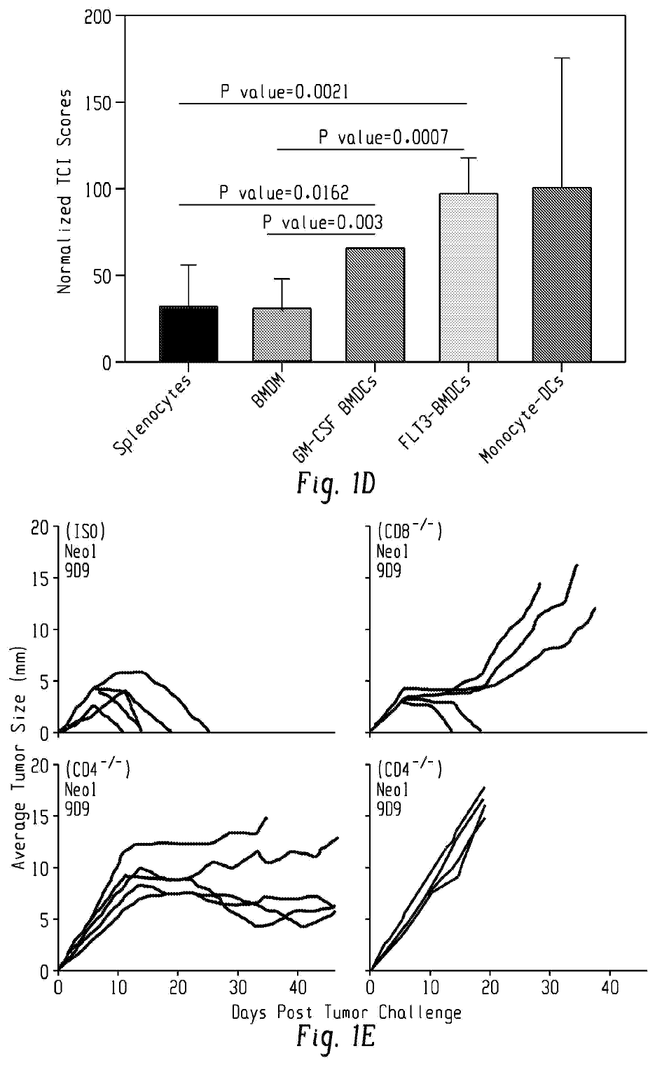

To determine the best cell-based vaccine strategy, we initially chose the well-characterized Meth-A fibrosarcoma tumor model and the defined Neo1 peptide neo-epitope in BALB/cJ mice. Prior to tumor challenge, mice were immunized twice, one week apart, with Neo-1 peptide-pulsed splenocytes (1.5×10⁷), bone marrow-derived macrophages (3×10⁶), GM-CSF-induced BMDCs (3×10⁶), or control treated (splenocytes alone without peptide). With the second immunization, half of the mice were also treated with anti-CTLA-4 (9D9 antibody) to block signals that would downregulate immune response. Mice were then challenged with 9.5×10⁴ Meth-A tumor cells subcutaneously, and tumor growth was measured weekly (FIG. 1A). Similarly, when mice were immunized with FLT-3-induced BMDCs (FIG. 1 B), with or without Neo1 peptide and with or without 9D9, the best response was observed in the mice receiving Neo1 plus 9D9 (FIG. 1 C right panel), though tumor regression was slower in these mice than in the mice that received GM-CSF-induced BMDCs. FIG. 1D compares total Tumor Control Index (TCI) for the groups of FIG. 1A. In this method—previously developed in our lab—the tumor rejection, tumor progression as well as tumor stability are parametrized and combined to yield total TCI score which reflects the inhibition of the tumor growth in each group. Based on total TCI scoring the best tumor response was observed in mice that received BMDCs and 9D9; these animals had complete resolution of tumors by two weeks in all mice. By contrast, no other treatment resulted in complete tumor response in all mice, and the mice that received Neo1-pulsed BMDCs alone had the best response of the animals not treated with 9D9. Moreover, the best approach would be one that is readily translatable to the clinic, since development of a perfect dendritic cell vaccine approach that cannot be used in the clinic would not be very useful. In order to translate these experiments, we harvested mouse blood monocytes and differentiated them to dendritic cells and used for immunization similar to FIG. 1 A. Again, the best response was observed in the mice receiving Neo1 plus 9D9. These data show that combination of DC therapy with a proper neoepitope may be useful in cancer immunotherapy clinical trials.

To assess the contributions of CD4 and CD8 T cells, a parallel cohort of mice was treated with BMDCs as in FIG. 1A, but pretreated with depleting antibodies specific for CD4 or CD8 before tumor challenge (FIG. 1 E). In these mice, tumors initially grew in all mice, as expected, and the growth was blunted in mice treated with 9D9. Immunization with Neo-1-loaded BMDCs induced tumor rejection, as before. Depletion of either CD8 or CD4 cells led to a modest blunting of the anti-tumor effect of the immunization. In fact, the tumors grew faster in CD8- and CD4-depleted mice than they did in control mice. In the group of mice that both CD4 and CD8 were depleted, all mice were sacrificed before day 20 due to the rapid tumor growth. Thus both CD8 and CD4 T cells are important for tumor rejection following immunization with peptide-loaded BMDCs.

### Example 2: BMDCs as Antigen Presenting Cells or Reservoir

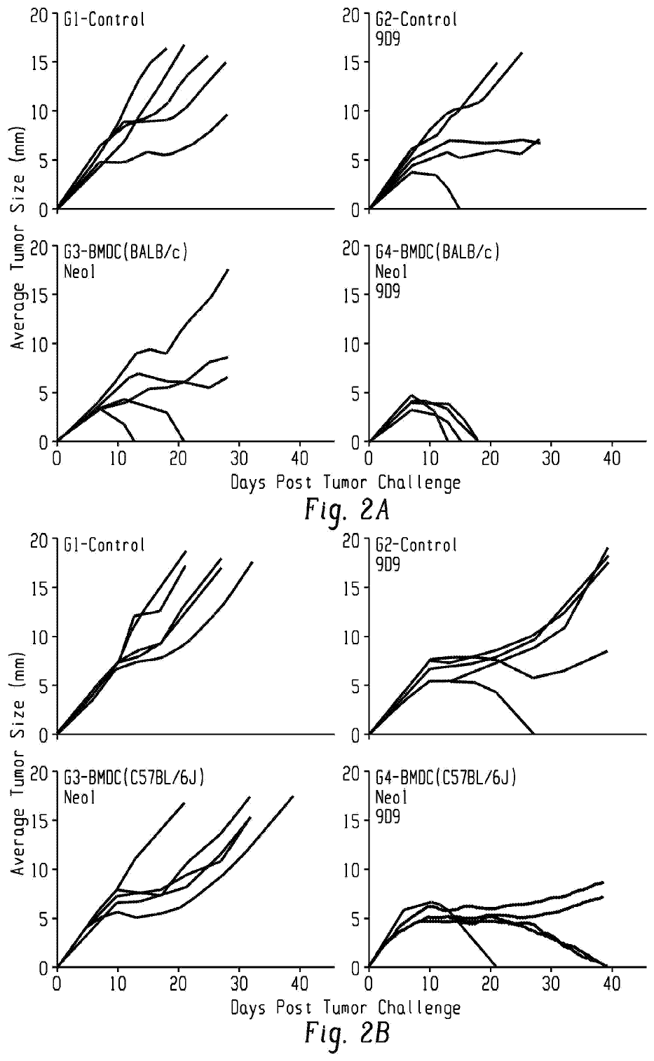

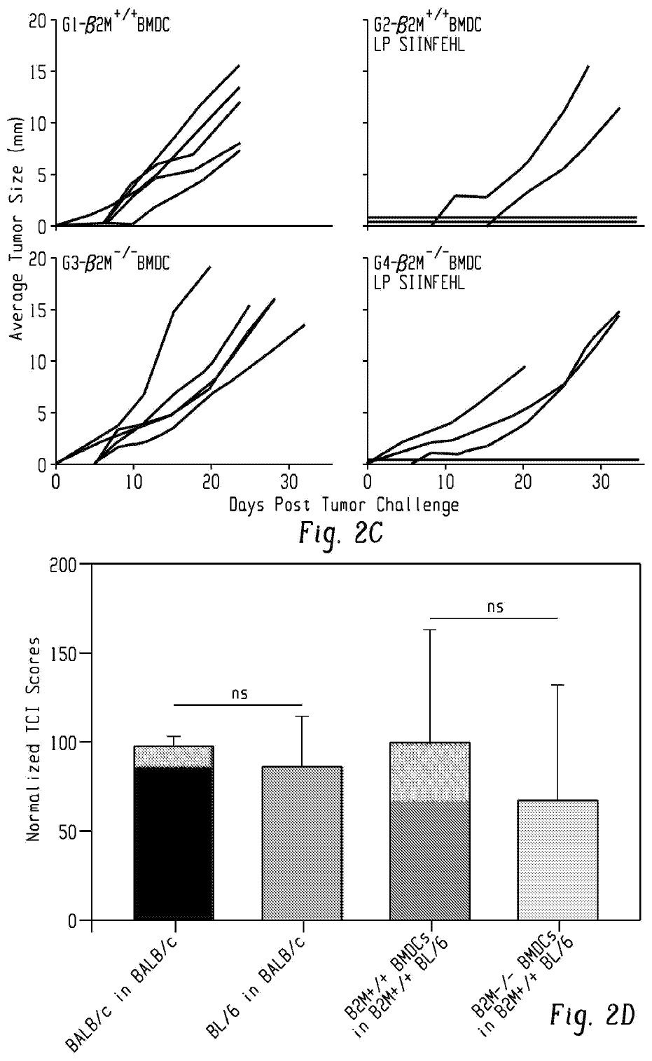

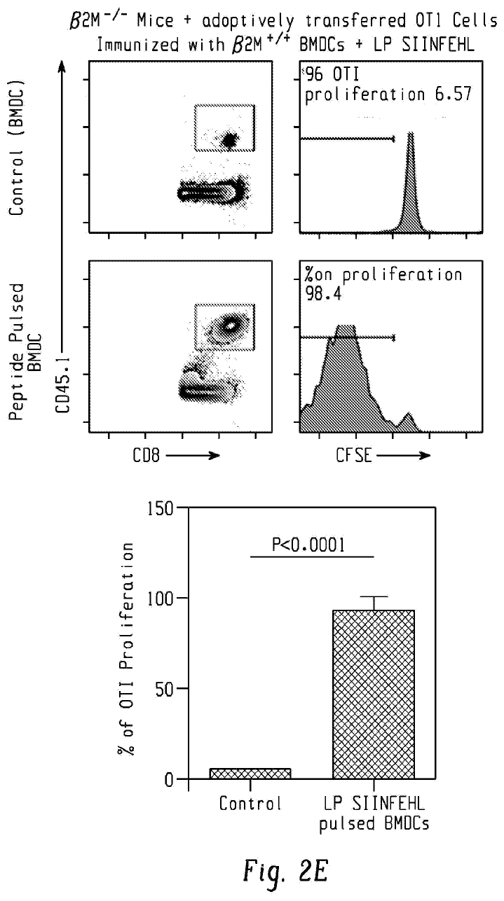

To assess whether the BMDCs were stimulating T cells directly to affect tumor clearance, we utilized MHC-mismatched BMDCs. BALB/cJ mice were immunized with BMDCs derived from either C57Bl/6 (H-2^{b}) (FIG. 2A) or BALB/cJ (H-2^{d}) (FIG. 2B) mice, with or without Neo1 peptide and with or without 9D9. As expected, the mice given BALB/cJ-derived BMDCs, Neo-1, and 9D9 rapidly rejected the Meth-A tumor challenge. Surprisingly, though, the mice given C57Bl/6 BMDCs, Neo-1, and 9D9 also rejected the Meth-A tumors, albeit over a longer time course, and less completely. These data suggested that the BMDCs might function not by stimulating T cells directly, but rather as a reservoir for immunizing peptides that were presumably re-presented by endogenous APCs.

To better understand the importance of direct antigen presentation by the BMDCs, we turned to the B16-OVA tumor model, which is syngeneic with C57Bl/6. First, to show that the model worked as expected, C57Bl/6 mice were immunized with BMDCs from C57Bl/6 mice, either with or without the OVA long peptide (LP SIINFEHL, 18-mers). Two weeks after the second immunization, the mice were challenged with 1.5×10⁵ B16-OVA tumor cells by subcutaneous injection. The peptide-loaded BMDCs mediated tumor rejection with this strong antigen (data not shown). We then created BMDCs from C57Bl/6 mice in which the 3-2 microglobulin gene had been genetically deleted. Cells from these mice lack MHC class I, and are unable to present the LP SIINFEHL peptide. Two immunizations with normal C57Bl/6 BMDCs or β-2 microglobulin^{-/-} C57Bl/6 BMDCs were performed 1 week apart. One week after the second immunization, mice were challenged with B16-OVA as above, and tumor growth measured (FIG. 2B). As expected, the mice treated with normal BMDCs rejected the tumors. Surprisingly, we also saw tumor rejection in the mice treated with β-2 microglobulin^{-/-} BMDCs, though it was less robust than that observed in the control mice. These data further suggest that the BMDCs act as a reservoir for peptide, which is then re-presented by endogenous APCs. FIGS. 2C and 2D show the normalized TCI scores of syngeneic (BALB/cJ), allogeneic (C57BL/6), β-2 microglobulin^{+/+} and β-2 microglobulin^{-/-} BMDCs immunization.

Although based on FIGS. 2C and D, the tumor rejection capacity of syngeneic (BALB/cJ) versus allogeneic (C57BL/6) BMDCs or β-2 microglobulin^{+/+} versus β-2 microglobulin^{-/-} BMDCs are not statistically significant due to the fact that this phenomenon was reproducible during repeated experiments, we still did investigate this difference further. In order to do so, OVA-specific CD8+ T cells were enriched from single cell suspension of spleen, mesenteric LN, and skin draining LNs of CD45.1 OT-I mice, using negative immunomagnetic selection (STEMCELL™ Technologies). OT-I CD8+ T cells were then labeled with 5 μM CFSE (Biolegend) and about 5×10⁵ labeled OT-I CD8+ T cells were adoptively transferred into two groups (n=3) of β2M −/− mice, on day −3. After 24 hours, mice of the control group and experimental group were intradermally immunized with BMDCs alone or BMDCs pulsed with longer version of SIINFEKL, respectively. Draining lymph nodes were harvested from individual mice, on day 0 (72 hrs after OT-I transfer) and dilution of CFSE in gated CD45.1+ OT-I CD8+ T cells was analyzed by flow cytometry (FIG. 2 E). These data clearly show that BMDCs can have a role in direct priming of CD8 T cells without the help of endogenous APCs and the difference between of tumor rejection potential of syngeneic (BALB/cJ) versus allogeneic (C57BL/6) BMDCs or β-2 microglobulin^{+/+} versus β-2 microglobulin^{-/-} BMDCs comes from APC role of BMDCs immunization.

### Example 3: CD11c⁺ MHCII^{lo/int} BMDCs Work the Best Among Different BMDCs Subpopulations

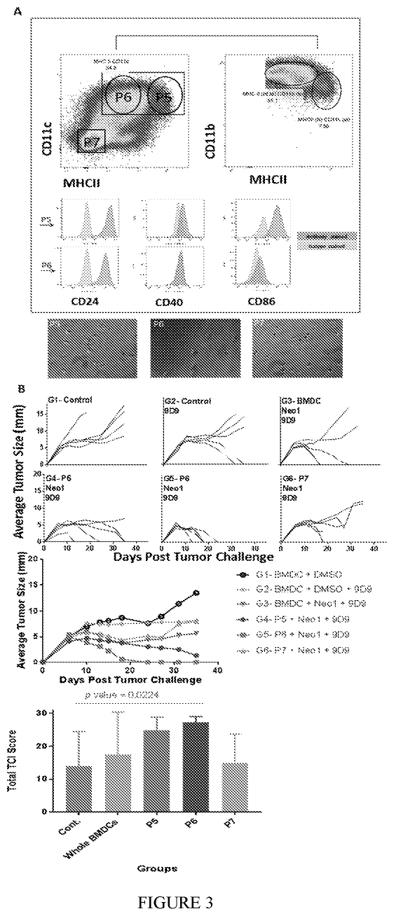

GM-CSF-induced BMDCs are a heterogeneous population of cells. To better characterize the cells responsible for tumor rejection following immunization with peptide-pulsed BMDCs, we sorted the BMDCs based on expression of CD11c and MHC class II into three populations (FIG. 3A): undifferentiated cells without CD11c and MHCII expression (P7), CD11c⁺ MCHII^{lo/int} (P6) and CD11c⁺ MHCII^{hi} (P5). These cells (5×10⁵/mouse) were then used to immunize mice as described above, and mice were challenged with Meth-A tumor cells one week after the second immunization. Anti-CTLA4 treatment was performed similar to FIG. 1 and tumor growth was measured twice a week. FIG. 3B middle and bottom panels show the group average of tumor growth and total TCI score for FIG. 3B groups, respectively. As expected, this lower dose of BMDCs mediated a less robust rejection of tumors, compared with the 3×10⁶ cells used in FIGS. 1 and 2. Cells without CD11c expression did not mediate significant tumor regression (FIG. 3A). Further analysis (FIG. 3 A, inset) showed that the MCHII^{lo} cells were uniformly high in CD11b expression, while the MHCII^{hi} cells consisted to two populations, one with high-level CD11b expression and the other low in CD11b. This population (P5) also had expression of the costimulatory molecules CD86, CD40, and CD24, which resembled mature BMDCs. While these co-stimulatory molecules were expressed on the MCHII^{lo} cells in a very low level, if any, which are representative of immature BMCDs. Of these cells, the MCHII^{lo} cells mediated much better tumor regression than did the MHCII^{hi} cells (FIG. 3A).

### Example 4: Further Analysis of CD11C⁺MHCII^{lo} GM-CSF Bone Marrow Derived Dendritic Cells

This example describes further details of some of the same experiments as described in Examples 1-3 and additional experiments.

Materials and Methods

Mice, Tumors and Peptides.

C57BL/6, BALB/cJ C57BL/6-Tg (TcraTcrb) 1100Mjb/J (OT-I Tg mice) and B6.129P2-B2mtmlUnc mice (6 week female) were purchased from the Jackson Laboratory, and maintained in virus free mouse facilities under approval from the Institutional Animal Care and Use Committee. CD45. 1+RAG^{-/-} OT-I TCR Tg mice (OT-I) were bred and housed in barrier facilities maintained by the Center for Laboratory Animal Care (CLAC). These mice have transgenic T cell receptor that is designed to recognize the complex of H2Kb and ovalbumin peptide residues 257-264. Meth A cells that have been in our lab since 1988, were originally obtained from Lloyd J. Old. Meth A ascites cells were used for passage. B16 melanoma cells that were permanently transfected with ovalbumin antigen (B16-OVA) were generously gifted by Dr. Nick Restifo (Center for Cancer Research, National Cancer Institute, Bethesda, Md., USA). Neo1 a neoepitope of the BALB/cJ Meth A fibrosarcoma, was synthesized by JPT PeptideTechnologies GmbH (Berlin, Germany).

Immunization.

Splenocytes, day 7 Granulocyte-macrophage colony-stimulating factor-derived BMDCs (GM-CSF-BMDCs), day 10 FMS-like tyrosine kinase 3 ligand BMDCs (FLT3L-BMDCs), bone marrow-derived macrophages (BMDMs) and day 3 monocyte-derived DCs (Mo-DCs) were pulsed with 40 μg/mouse Neo1 (1 μl of a 100 μM Neo1 peptide solution was added to 7.5 million BMDM, BMDC or 500,000 MO-DCs in 200 μl RPMI medium) or chicken ovalbumin-derived long peptide 18-mer (LP) LEQLKSIINFEHLKEWTS (SEQ ID NO: 3) (referred to as LP SIINFEHL) for 2 hours. The LP contains the dominant K^{b}-restricted epitope SIINFEHL within it. The natural epitope is SIINFEKL; however, SIINFEHL has been shown to be equivalent with respect to its interaction with the T cell Receptors as known in the art. The LP, as opposed to the precise peptide, is used because it gives more consistent results as known in the art. All immunizations were carried out in presence of CTLA4 blockade, using the IgG2b antibody (9D9), administered with the second immunization and every three days after tumor challenge. We have demonstrated that certain neoepitopes demonstrate their fullest activity only in combination with CTLA4 blockade.

T Cell Depletion.

BALB/cJ mice were injected with 250 μg of ISO (isotype control antibody), CD8 (Rat IgG2b, clone 2.43) or CD4 (Rat IgG2b, clone GK1.5) depletion antibodies 2 days before each immunization and tumor challenge and every week afterwards.

BMDCs and BMDMs.

Bone marrow cells (2-3 million per ml) of 6-8 week old mice were cultured in complete RPMI supplemented with 20 ng per ml recombinant murine GM-CSF (Peprotech) and incubated at 37° C. for 7 days to generate GM-CSF-BMDCs. Bone marrow cells (10 million per ml) of 6-8 week old mice were cultured in complete RPMI supplemented with 200 ng per ml recombinant murine FLT3L (TONBO biosciences; San Diego, Calif.) and incubated at 37° C. for 10 days to generate FLT3L-BMDCs.

BMDMs were generated by flushing femurs and tibias with DMEM. Cells were incubated overnight in 25 cm²-flasks at 37° C. The following day, 10⁷ of the suspended cells were harvested and maintained in 10 cm² bacteriological Petri dishes (BD-Falcon) with DMEM supplemented with 10%0/FBS, 20% L929-cell conditioned media (supernatant of L929 cells which contains Macrophage colony-stimulating factor (M-CSF)) and supplements. Cultures were fed with five ml of the medium after three days. Seven days after the isolation, cell monolayers (BMDMs) were exposed to ice-cold PBS and recovered by scraping.

Mo-DCs.

Peripheral blood mononuclear cells (PBMCs) were isolated using lymphoprep and SepMate™ from StemCell Technologies Co. (Vancouver, Canada). Monocytes were isolated from PBMCs using EasySep™ Mouse Monocyte Isolation Kit. 500000 monocytes/ml were cultured in complete RPMI with FBS supplemented with 50 ng/ml GM-CSF (Peprotech; Rocky Hill, N.J.) plus 25 ng/ml IL-4 (StemCell Technologies) in 24-well plates for 3 days.

Flow Cytometry.

The antibodies specific for FITC CD1 Ic (clone N418), Fixable Viability Dye eFluor® 780 and PE CD3 (clone 145-2C11) were purchased from eBioscience (Thermo Fisher Scientific; Carlsbad, Calif.). The antibodies specific for PerCP MHCII (clone M5/114,15.2), Pacific Blue™ F4/80 (clone BM8), APC CD49b (clone DX5), FITC CD4 (clone RM4-5), Pacific Blue™ B220 (clone RA3-6B2) and PE/cy7 CD19 (clone 6D5) were purchased from Biolegend (San Diego, Calif.). V500 CD11b (clone M1/70), APC CD86 (clone GLl[RUO]), CD40 (clone 3/23) and CD24 (clone M1/69) were purchased from BD Bioscience (San Jose, Calif.). VioGreen™ CD8 (clone 53-6.7) was purchased from Miltenyi Biotec (Bergisch Gladbach, Germany). Fluoresbrite® plain YG 0.5 micron microspheres (2.5% solid-latex) were purchased from Polysciences, Inc. (Warrington, Pa.). Flow cytometry was performed using Miltenyi Biotec MACSQuant® analyzer and ImageStream®X Mark II Imaging Flow Cytometer. Cell sorting was accomplished with BD LSR II-B. Analysis was done using FlowJo software.

Total mRNA Sequencing.

Sequencing of cDNA was performed by the Illumina NextSeq™ 500 Sequencing System (Illumina; San Diego, Calif.). RNA-Seq paired-end reads were aligned to the Ref Seq Release 77 mouse transcriptome reference using HISAT2 as known in the art. IsoEM2, an expectation-maximization algorithm for inference of isoform- and gene-specific expression levels from RNA-Seq data, was used to estimate gene expression levels. Gene expression was reported as Transcripts per Million (TPM) units. Each gene was assigned the value of log 2 (TPM+1) to generate heat maps. IsoDE2 method was run for gene differential expression. Differential expressed genes (DEGs) were investigated by the Ingenuity pathway analysis (IPA) software program, which can analyze the gene expression patterns using a scientific literature based database (Qiagen; Hilden, Germany). The Web-based tool Morpheus was used to generate heat maps of genes with assigned value of log 2 (TPM+1).

Statistical Analysis:

P-values for TCI scores comparisons were calculated using a two-tailed t-test, using GraphPad Prism 5.0 (GraphPad; La Jolla, Calif.). P<0.05 was considered statistically significant.

The results of the experiments are described below.

Dendritic Cells but not Macrophages Mediate Potent Neoepitope-Elicited Tumor Protection.

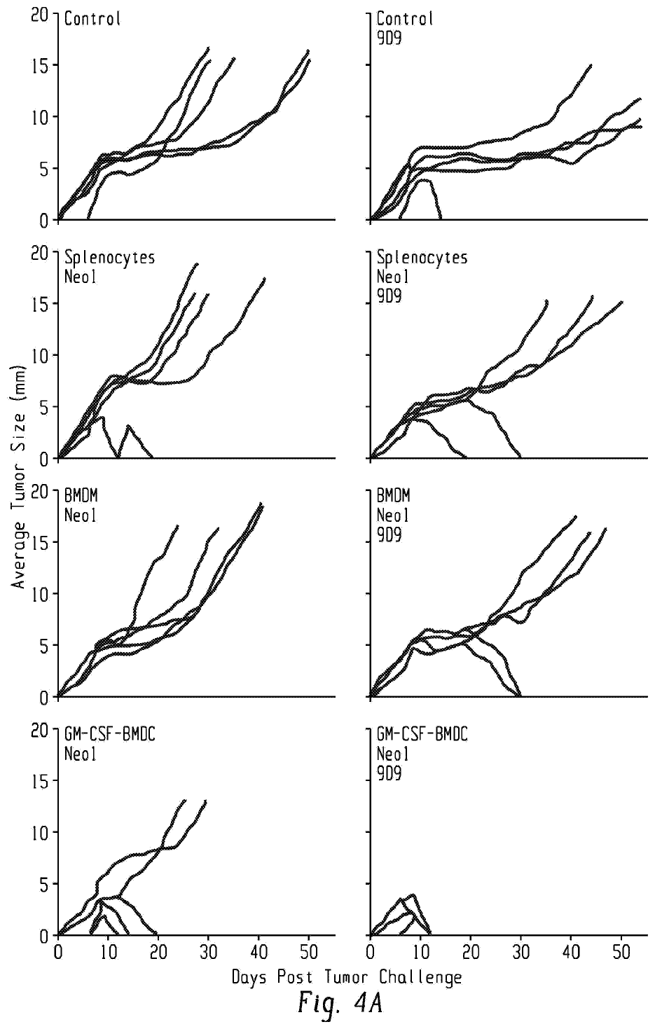

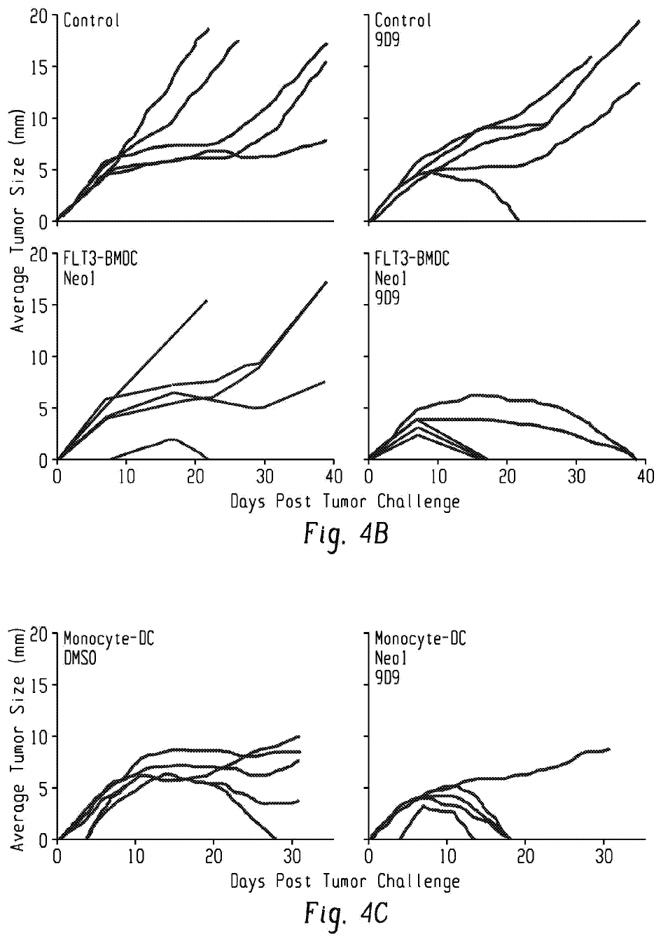

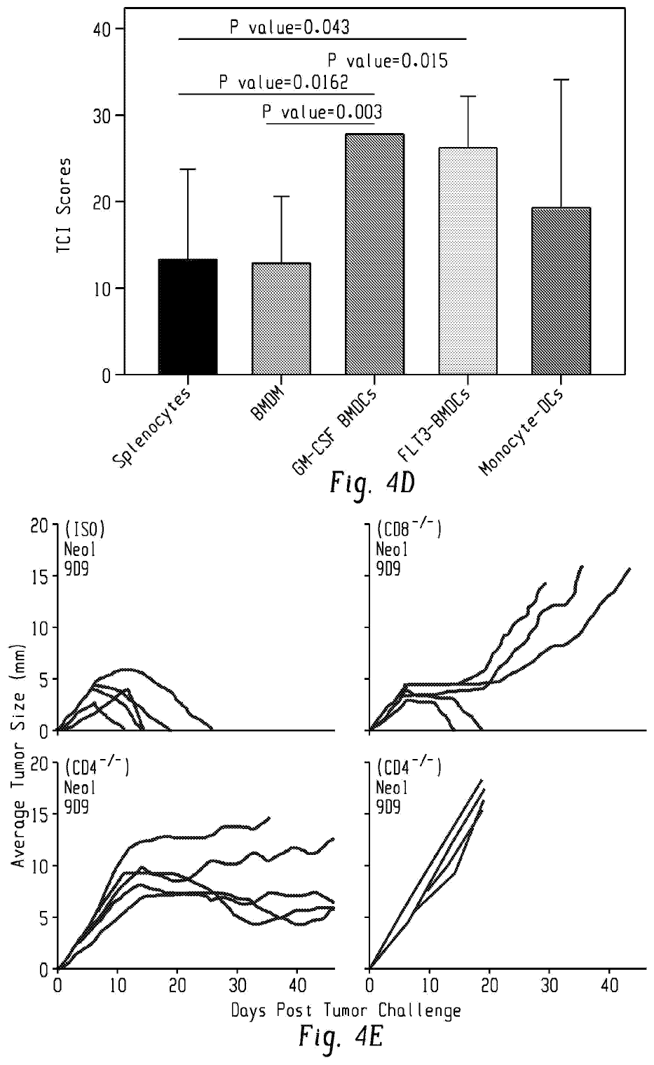

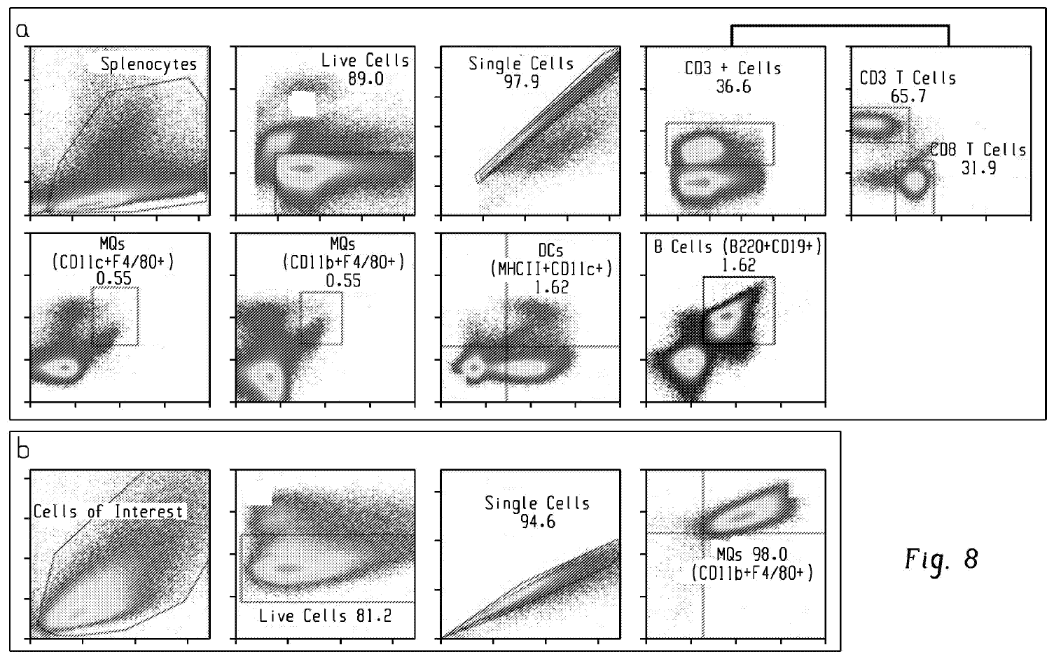

The mutant neoepitope Neo1 of the Meth A fibrosarcoma of BALB/cJ mice was used as the antigen. Mice were immunized twice, one week apart, with Neo1-pulsed splenocytes (1.5×10⁷), BMDM (3×10⁶), or GM-CSF-BMDCs (3×10⁶) as adjuvants. All mice were challenged with 9.5×10⁴ Meth A tumor cells subcutaneously, and tumor growth was measured twice a week (FIG. 4A, left panels). The DC-Neo1 immunized mice were the only group which showed tumor protection (⅗ mice complete protection). Specifically, Neo1-pulsed macrophages immunization did not elicit any antitumor activity. Splenocytes and BMDM were characterized using antibody markers for macrophages, DCs, B cells, CD4 and CD8 T cells (FIG. 8).

The same immunization was attempted in the presence of anti-CTLA4 antibody (9D9 IgG2b) (FIG. 4A, right panels). The isotype control antibody (mouse IgG2b isotype control) did not have any effect on tumor rejection in over ten experiments. The best tumor rejection (100%) was observed in mice immunized with BMDCs. The tumor rejection capacity of different immunization methods was quantified and statistically compared them using Tumor Control Index (TCI) scores (Corwin et al., J. Immunol. Methods 445:71-76 (2017)). The TCI score parametrizes and combines the tumor inhibition, tumor rejection, as well as tumor stability scores to yield a total TCI score which reflects the inhibition of the tumor growth in each group. TCI score of the GM-CSF-BMDC group was significantly higher than that of macrophages (P=0.003) and splenocytes (P=0.0162) groups (FIG. 4D).

Other types of DCs were also tested as adjuvants in the same setting of tumor rejection as in FIG. 4A. Mice were immunized with FLT3L-BMDCs, with or without Neo1 peptide and with or without 9D9 IgG2b (FIG. 4B). Complete (100%) rejection was observed in mice immunized with Neo1-pulsed FLT3L-BMDCs in the presence of 9D9 IgG2b (FIG. 4B). TCI score of FLT3L-BMDC group was significantly higher than that of macrophages (P=0.015) and splenocytes (P=0.043) groups (FIG. 4D). In order to test the adjuvanticity of Mo-DCs, mouse blood monocytes were harvested and differentiated to dendritic cells with GM-CSF and 1L-4 and used for immunization. Four out of five Meth A tumors were rejected in the mice immunized with Mo-DCs (FIG. 4C). The adjuvanticities of GM-CSF BMDC, FLT3L BMDC and Mo-DC were statistically indistinguishable from each other (FIG. 4D). However, GM-CSF BMDC was considered to be the better adjuvants because the kinetics of tumor rejection seen in BMDC-immunized mice is clearly very different from that seen with Mo-DCs and FLT3L-DC (see FIG. 4A, 4B, 4C). All mice underwent complete rejection in case of BMDC-immunized mice within 10-12 days post challenge. In Mo-DCs and FLT3L-DC-immunized mice tumor rejection occurred over 17-40 days (FLT3-DC), or 17-20 days (Mo-DCs) (FIG. 4B). There was almost no variability in tumor rejection in BMDC-immunized mice as seen in many of the figures.

CD8 and CD4-dependence of immunity elicited by Neo1-pulsed BMDC and 9D9 IgG2b immunization was tested by depleting mice of the respective cells as described in Methods. Tumor protection was observed to be CD8, as well as CD4 dependent (FIG. 4E).

Adjuvanticity of GM-CSF-BMDCs Derives from their Role of Antigen Donor Cells as Well as Antigen Presenting Cells.

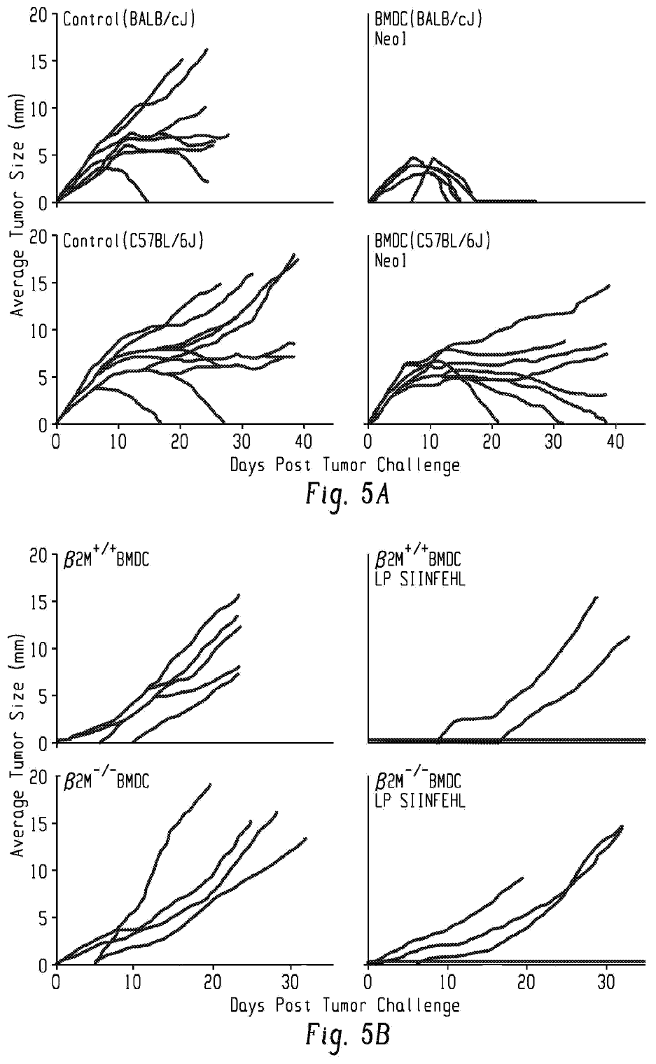

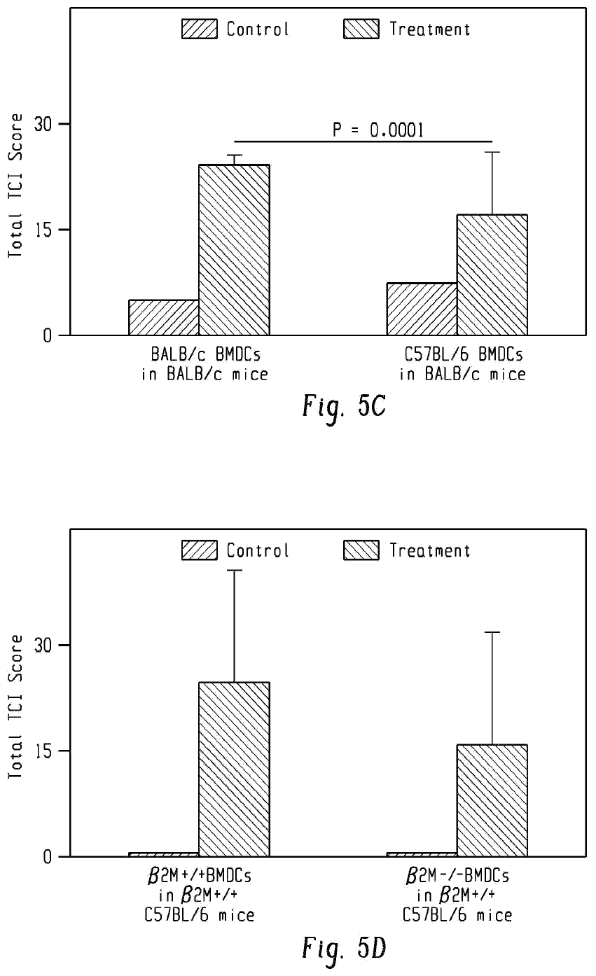

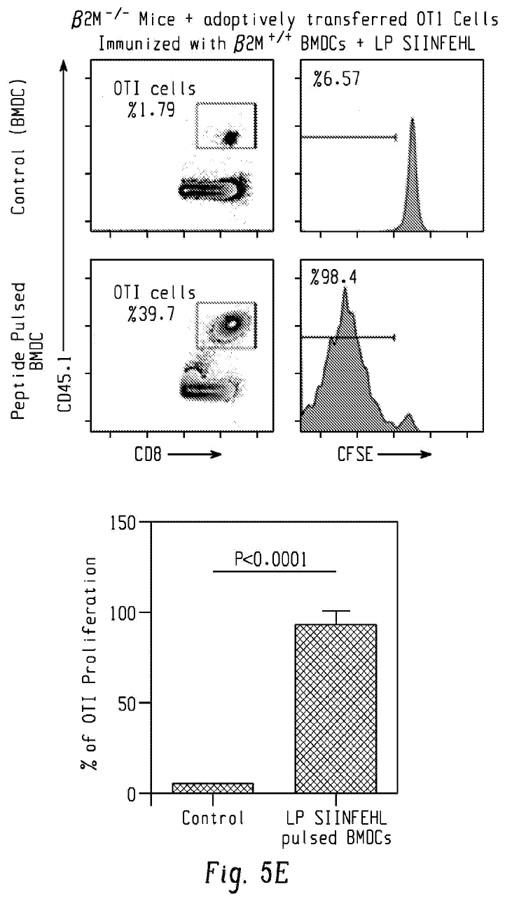

In order to dissect the role of BMDCs as ADCs versus APCs, BMDCs of two different haplotypes were used as adjuvant. BALB/cJ mice were immunized with BMDCs derived from BALB/cJ (H-2^{d}) or C57BL/6 (H-2^{b}) mice, with or without Neo1 peptide and with 9D9 IgG2b (FIG. 5A). The mice immunized with BALB/cJ-derived BMDCs, Neo1, in the presence of 9D9 IgG2b rejected the Meth A tumor challenge completely (100%) and rapidly (within <20 days). Mice immunized with C57BL/6 BMDCs, Neo1 also rejected the Meth A tumors, albeit less completely (50%) and over a longer time course (between 20 and >40 days). Normalized TCI score of mice immunized with Neo1-pulsed isogeneic BMDCs is significantly higher (P=0.0001) than in mice immunized with Neo1-pulsed allogeneic BMDCs (FIG. 5C). The higher tumor protection in the mice immunized with Neo1-pulsed (syngeneic) BALB/cJ BMDCs may be due to the APC function of the syngeneic BMDCs (i.e., BMDCs with self MHC may serve both as antigen reservoirs as well as APCs).

In order to further dissect the difference between ADC and APC roles of BMDCs, MHC I-expressing and non-expressing DCs from β2 microglobulin^{+/+} (β2M^{+/+}) or β2M^{-/-} mice were used. β2M^{-/-} are available only in the C57BL/6 and not the BALB/c background where Neo1 may be used. For this specific purpose, the experiment was switched to the use of the chicken ovalbumin (and its well-known, dominant K^{b}-restricted epitope SIINFEKL) for this experiment only. C57BL/6 mice were immunized with LP SIINFEHL (18-mer) pulsed (β2M^{+/+} or β2M^{-/-} C57BL/6 BMDCs twice, one week apart. (β2M^{-/-} DCs can act only as ADCs and not as APCs.) One week after the second immunization, mice were challenged with 1.5×10⁵ B16-OVA tumor cells and tumor growth was measured (FIG. 5B). Mice immunized with normal BMDCs (β2M^{+/+}) showed tumor protection (60% complete protection) while mice immunized with β2M^{-/-} BMDCs showed less robust tumor protection (40% complete protection). Normalized TCI score of the mice immunized with Neo1-pulsed β2M^{+/+} BMDCs was higher than that of the mice immunized with Neo1-pulsed β2M^{-/-} BMDCs (FIG. 5D), although the difference was not statistically significant in and of itself. However, a statistically highly significant difference (P=0.0001) between syngeneic and allogeneic BMDCs was seen in the tumor rejection data (FIG. 5A, 5C).

A more stringent and quantitative test for the role of BMDCs as APCs was devised. OVA-specific CD8⁺ T cells were enriched from single cell suspension of spleen, mesenteric lymph node (LN), and skin draining LNs of CD45.1 OT-I mice. OT-I CD8⁺ T cells were then labeled with CFSE, and the labeled OT-I CD8⁺ T cells were adoptively transferred into two groups of β2M^{-/-} mice (β2M^{-/-} mice have no competent APCs). Mice were immunized with LP SIINFEHL-pulsed β2M^{+/+} BMDCs. Draining LNs were harvested from individual mice, and dilution of CFSE in gated CD45.1⁺ OT-I CD8⁺ T cells was analyzed (FIG. 5E). β2M^{-/-} mice immunized with 32M+/+DCs supported vigorous proliferation of OTI cells (p<0.0001) indicating that the immunizing BMDCs acted as APCs in β2M^{-/-} mice (FIG. 5E bottom panel). β2M^{+/+} mice immunized with OVA in any form always supported vigorous proliferation of OTI cells.

CD11c⁺ MHCII^{lo} GM-CSF-BMDCs Mediate the Most Potent Neoepitope-Elicited Tumor Protection.

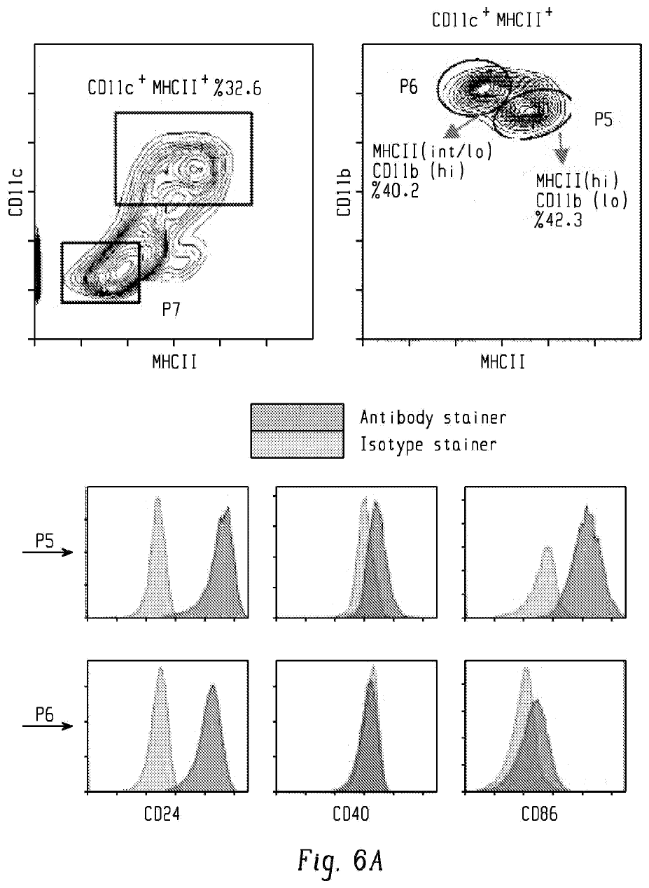

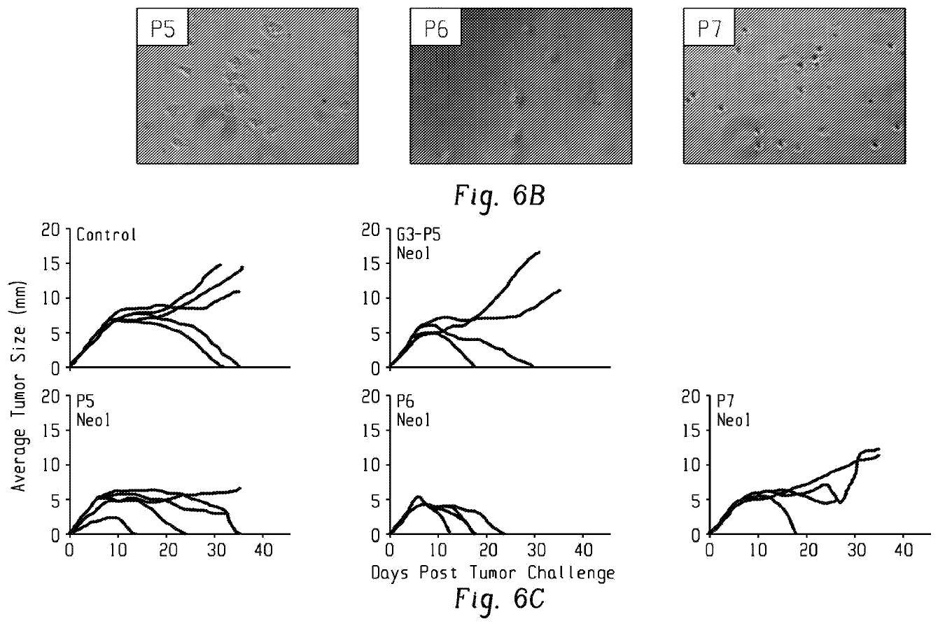

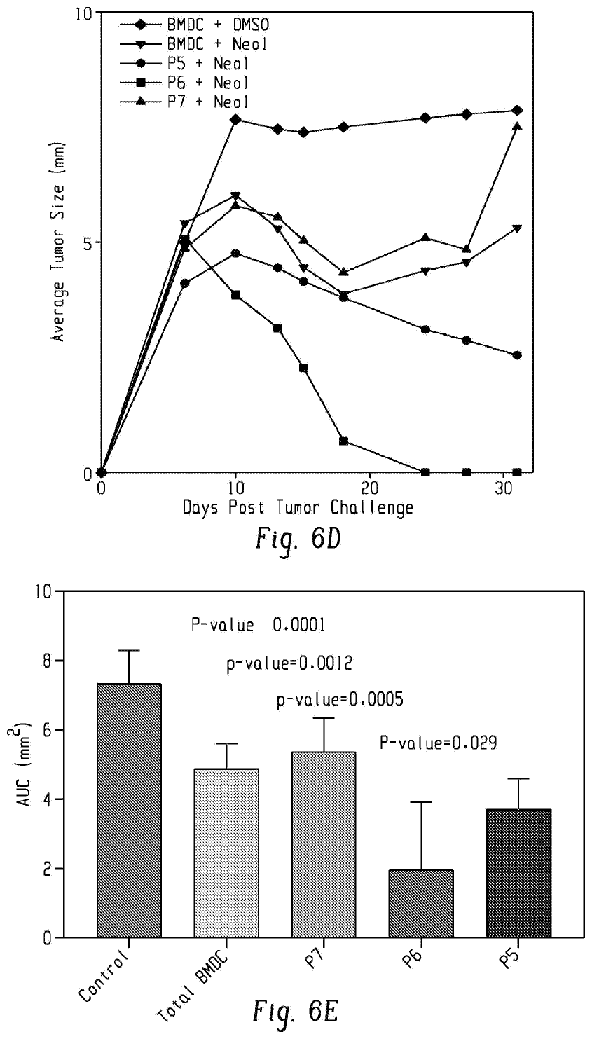

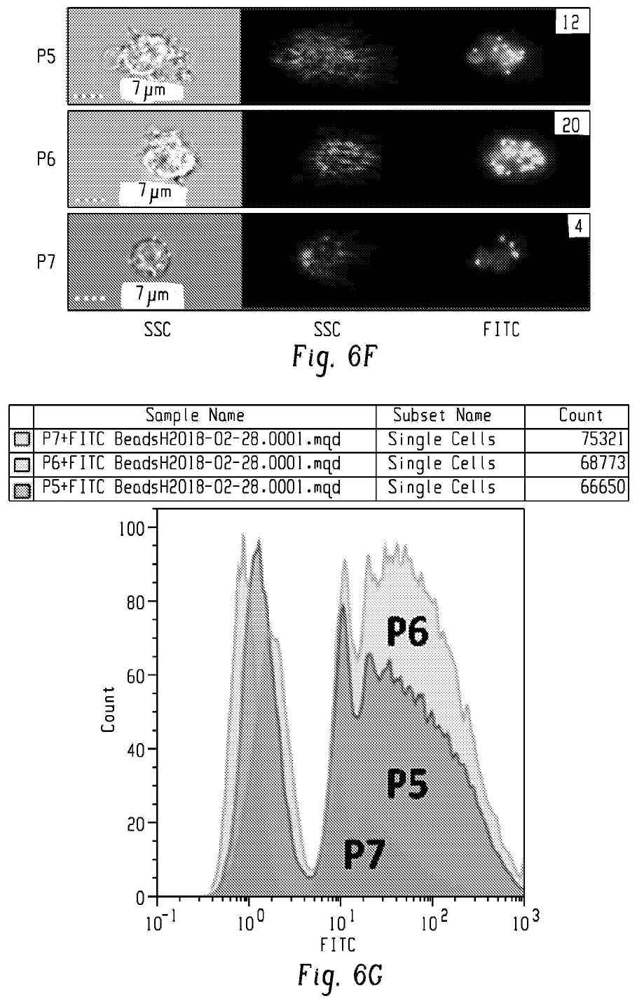

BMDCs were sorted based on expression of CD11c and MHC class II into three sub-populations similar to the sorting strategy adopted earlier (FIG. 6A): undifferentiated cells without CD11c and MHCII expression (P7), CD11c⁺ MCHII^{lo} cells (P6) and CD11c⁺ MHCII^{hi} cells (P5). P5 and P6 sub-populations were also characterized for the expression of CD24, CD40 and CD86 co-stimulatory molecules as well CD11b (FIG. 6A bottom panels). P5, P6 and P7 cells were also analyzed by light microscopy (FIG. 6B). Between P5 and P6 sub-populations, P5 showed higher expression of co-stimulatory molecules, lower expression of CD11b and a larger number of dendrites per cell. Hence, P5 resembled mature DCs and P6 appeared to have characteristics of immature DCs. This conclusion is also consistent with the expression of various surface markers as deduced by RNA sequencing analysis that was used to characterize immature and mature DCs. Table 1 shows that the P5 sub-population shows high expression of CD40, CD24, CD80/86 and MHCII as compared to the P6 sub-population (Table 1).

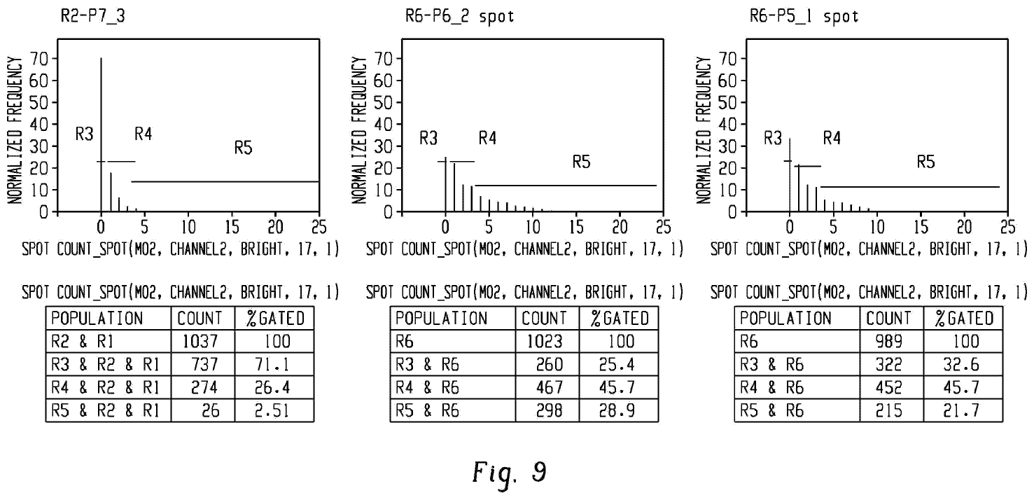

Mice were immunized with Neo1 pulsed P5, P6, P7 or whole BMDCs as control (5×10⁵/mouse) in the presence of 9D9 and challenged as in FIG. 4. A lower dose of BMDCs (5×10⁵/mouse) was used deliberately, so as to be able to see the activity in a titratable range. Indeed, at this dose of total BMDCs, significant but less robust rejection of tumors was seen, compared with that observed in FIGS. 4 and 5, where a higher dose of BMDCs (3×10⁶/mouse) was used (FIG. 6C). The P7 sub-population showed no adjuvanticity (p=0.593). However, the highest and highly significant adjuvanticity was observed in the mice immunized with the P6 sub-population where all mice (5/5) showed complete tumor regression with a rapid kinetics (P5 compared with P6, P-0.029). Data with individual mice are shown in FIG. 6C and pooled data from each group in FIG. 6D. Area under the curve values of the mice immunized with P6 sub-population is statistically significantly lower than the corresponding values in mice immunized with P5 and P7 sub-populations (FIG. 6E). Hence, P6 yielded better tumor rejection than P5 and P7 sub-populations. The sorted P5, P6 and P7 sub-populations were incubated with 0.5 micron FITC microspheres for 30 min to test the capacity of antigen uptake of each sub-population. Cells were thoroughly washed to remove excess beads from the cell surface. Using ImageStream®X Mark II Imaging Flow Cytometer (FIG. 6F) and MACS Quant® (FIG. 6G) the number of beads taken up by each sub-population was quantified. The highest number of beads was observed in the P6 sub-population. Around 71%, 25% and 32% (of P7, P6 and P5 cells, respectively) were observed to not have any beads. The group that was able to take up the highest percentage of more than 3 beads was P6 (28.9%) while the P5 and P7 percentages were 21.7 and 2.51%, respectively (FIG. 9).

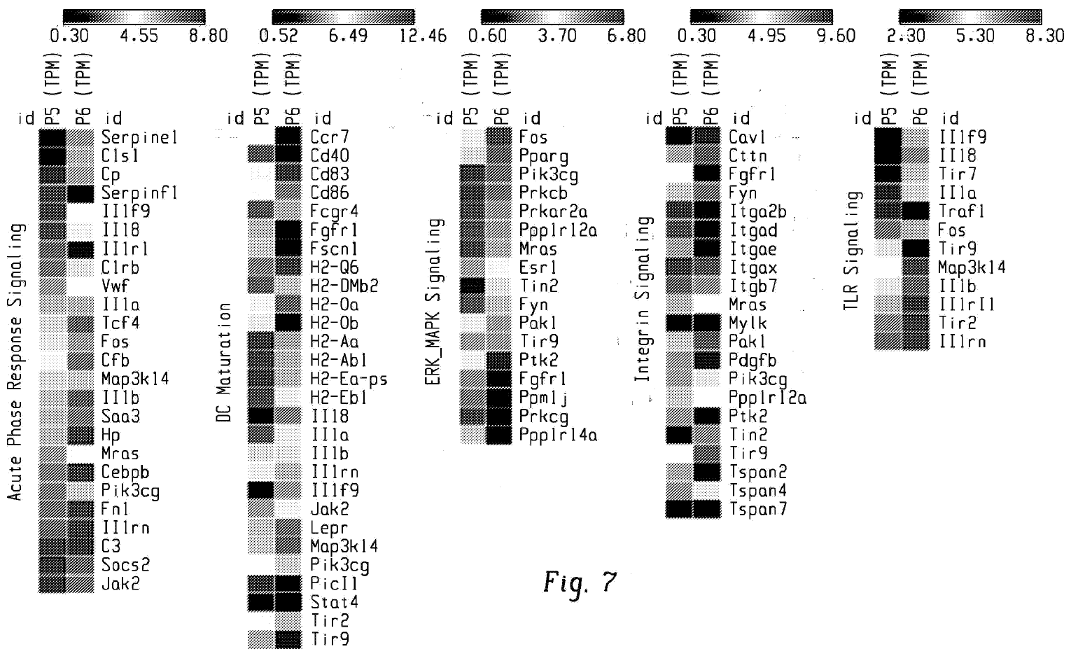

Using total mRNA sequencing, differential gene expression analysis was performed on P5 and P6 sub-populations. Both sub-populations showed RNA expression signatures for DCs as well as macrophages, although the P5 sub-population showed higher expression level of all markers tested. P5 and P6 sub-populations were compared for maturation phenotype using RNASeq (Table 1). There are other significant transcriptional differences between the P5 and P6 sub-populations as well (Table 2). The expression of CD91 and LOX1, both heat shock protein receptors as well as two mannose receptors and selected toll like receptors (TLR1, TLR2, and TLR6) are increased in P6 as compared to P5. The increase is more substantial for some (LOX1, CD91 and TLR2) than for other genes. CD36 (scavenger receptor) is the only major receptor that is substantially reduced in P6 as compared to P5 (>4 fold). The heat maps of the transcriptional data (FIG. 7) show that the P5 sub-population expresses a higher level of genes involved in DC maturation, migration (integrin signaling) and proliferation (ERK/MAPK signaling) while pathways involved in TLR signaling and acute phase response signaling predominates in P6 sub-population. The individual genes of each pathway that show the most difference between the P5 and P6 sub-populations are shown in Tables 3 and 4.

TABLE 1
Expression of transcripts for selected surface markers
on P5 and P6 cell sub-populations.

| | | | | Log₂Fold |
|---|---|---|---|---|
| | | P5 | P6 | Change |
| Surface Markers | Genes | (TPM^{a}) | (TPM^{a}) | (P6/P5)^{b} |
| CD209a | Cd209a | 146 | 6 | −4.6 |
| CD24a | Cd24a | 549 | 323 | −0.8 |
| CD80 | Cd80 | 32 | 16 | −0.9 |
| CD40 | Cd40 | 10 | 2 | −2.5 |
| CD86 | Cd86 | 109 | 16 | −2.7 |
| Histocompatibility 2, class II | H2-Ab1 | 3738 | 325 | −3.5 |
| antigen A, beta 1 | | | | |
| Histocompatibility 2, class II | H2-Aa | 5454 | 393 | −3.8 |
| antigen A, alpha | | | | |
| Histocompatibility 2, M region | H2-M2 | 3 | 0 | −3.8 |
| locus 2 | | | | |

^{a}Transcripts per Million
^{b}The Log₂ Fold Change of P5/P6 was computed using IsoDE2 tool with a statistical significance level of 0.05 (toolshed.g2.bx.psu.edu/view/saharlcc/isoem2_isode2/)

TABLE 2
Transcriptional profile of selected receptors in
P5 and P6 cell sub-populations.

| | | | | Log₂ Fold |
|---|---|---|---|---|
| | | P5 | P6 | Change |
| Protein name | Genes | (TPM^{a}) | (TPM^{a}) | (P6/P5)^{b} |
| CD91 | Lrp1 | 32.40 | 66.64 | 1.04 |
| LOX-1 | Olr1 | 4.38 | 21.75 | 2.31 |
| Mannose Receptor C-Type 1 | Mrc1 | 184.00 | 319.81 | 0.79 |
| Macrophage scavenger | Msr1 | 221.37 | 347.86 | 0.65 |
| receptor 1 | | | | |
| TLR1 | Tlr1 | 4.55 | 8.88 | 0.94 |
| TLR2 | Tlr2 | 99.55 | 260.73 | 1.38 |
| TLR6 | Tlr6 | 14.08 | 22.81 | 0.68 |

^{a}Transcripts per Million
^{b}The Log₂ Fold Change of P5/P6 was computed using IsoDE2 tool with a statistical significance level of 0.05 (toolshed.g2.bx.psu.edu/view/saharlcc/isoem2_isode2/)

TABLE 3
Pathways that are significantly upregulated in P6 compared to P5
sub-populations by IPA.

| | | Log₂ Fold | P5 | P6 |
|---|---|---|---|---|
| Pathways | Genes | Change (P6/P5) | (TPM^{a}) | (TPM^{a}) |
| Acute Phase | C3 | 1.1 | 335.2 | 747 |
| Response | C1rb | 1.4 | 5.9 | 15.4 |
| Signaling | C1s1 | 2.9 | 1.3 | 10 |
| (25/158 genes) | Cebpb | 1.7 | 109 | 349.8 |
| | Cfb | 2.1 | 27.7 | 123.8 |
| | Cp | 1.4 | 3.3 | 8.7 |
| | Fn1 | 1.3 | 123.3 | 307.4 |
| | Fos | 2.2 | 15.6 | 74.3 |
| | Hp | 2.5 | 54.9 | 304.4 |
| | Il18 | 2.1 | 4.1 | 17.6 |
| | Il1a | 2 5 | 10.5 | 58.3 |
| | Il1b | 1.6 | 51.2 | 155.9 |
| | Il1r1 | −1.1 | 5.3 | 2.6 |
| | Il1rn | 1.1 | 136 | 298.8 |
| | Il1f9 | 2.5 | 4 | 23.3 |
| | Jak2 | −1.8 | 439.4 | 125.6 |
| | Map3k14 | −1.7 | 40.5 | 12.1 |
| | Mras | −1.6 | 78.1 | 26.2 |
| | Plk3cg | −1.3 | 110.3 | 45 |
| | Saa3 | 1.1 | 54.6 | 116.8 |
| | Serpine1 | 2.6 | 1.2 | 7.9 |
| | Serpinf1 | −3.7 | 3.8 | 0.3 |
| | Socs2 | −1.5 | 390.7 | 139.3 |
| | Tcf4 | −1.4 | 14.2 | 5.3 |
| | Vwf | 1.4 | 8.2 | 22.4 |
| TLR Signaling | Fos | 2.2 | 15.6 | 74.3 |
| (12/72 genes) | Il18 | 2.1 | 4.1 | 17.6 |
| | Il1a | 2.5 | 10.5 | 58.3 |
| | Il1b | 1.6 | 51.2 | 155.9 |
| | Il1rl1 | −2.7 | 73.1 | 11.1 |
| | Il1rn | 1.1 | 136 | 298.8 |
| | Il1f9 | 2.5 | 4 | 23.3 |
| | Map3k14 | −1.7 | 40.5 | 12.1 |
| | Tlr2 | 1.4 | 107.6 | 282.3 |
| | Tlr7 | 1.6 | 7.9 | 24.8 |
| | Tlr9 | −2.4 | 29.7 | 5.5 |
| | Traf1 | −1.4 | 10.6 | 4 |

^{a}Transcripts per Million

TABLE 4
Pathways that are significantly downregulated in P6 compared to
P5 sub-populations by IPA.

| | | Log₂ Fold | | |
|---|---|---|---|---|
| Pathways | Genes | Change (P6/P5) | P5 (TPM^{a}) | P6 (TPM^{a}) |
| Dendritic Cell | Ccr7 | −4.5 | 79.1 | 3.5 |
| Maturation | Cd40 | −2.5 | 10.4 | 1.8 |
| (28/168 genes) | Cd83 | −3.2 | 74.2 | 7.8 |
| | Cd86 | −2.7 | 109.3 | 16.4 |
| | Fcgr4 | 1.5 | 11.1 | 31.7 |
| | Fgfr1 | −4.1 | 34.2 | 2 |
| | Fscn1 | −5.1 | 40.1 | 1.2 |
| | H2-Q6 | −1.1 | 17.2 | 8 |
| | H2-DMb2 | −2.6 | 1496 | 242.9 |
| | H2-Oa | −2.5 | 65.7 | 11.6 |
| | H2-Ob | −4.6 | −60.4 | 2.1 |
| | H2-Aa | −3.8 | 5337.3 | 385.1 |
| | H2-Ab1 | −3.5 | 3715.8 | 323.6 |
| | H2-Ea-ps | −4.1 | 5228.8 | 294.1 |
| | H2-Eb1 | −4.2 | 2511.4 | 135.4 |
| | Il18 | 2.1 | 4.1 | 17.6 |
| | Il1a | 2.5 | 10.5 | 58.3 |
| | Il1b | 1.6 | 51.2 | 155.9 |
| | Il1rn | 1.1 | 136 | 298.8 |
| | Il1f9 | 2.5 | 4 | 23.3 |
| | Jak2 | −1.6 | 439.4 | 125.6 |
| | Lepr | −1.5 | 39.1 | 13.7 |
| | Map3k14 | −1.7 | 40.5 | 12.1 |
| | Pik3cg | −1.3 | 110.3 | 45 |
| | Plcl1 | −1.2 | 7.3 | 3.1 |
| | Stat4 | −3.4 | 4.1 | 0.4 |
| | Tlr2 | 1.4 | 107.6 | 282.3 |
| | Tlr9 | −2.4 | 29.7 | 5.5 |
| Integrin Signaling | Cav1 | 1.6 | 1 | 3.2 |
| (20/211 genes) | Cttn | −1.1 | 11.9 | 5.4 |
| | Fgfr1 | −4.1 | 34 2 | 2 |
| | Fyn | −3.1 | 62.3 | 7.1 |
| | Itga2b | −1.1 | 4 | 1.9 |
| | Itgae | −2.6 | 11.1 | 1.8 |
| | Itgax | −1.2 | 789.5 | 328.4 |
| | Itgb7 | −1.1 | 261.3 | 125.1 |
| | Mras | −1.6 | 78.1 | 26.2 |
| | Mylk | −3.6 | 2.6 | 0.2 |
| | Pak1 | −1.5 | 15.8 | 5.6 |
| | Pdgfb | −1.7 | 10.3 | 3.1 |
| | Pik3cg | −1.3 | 110.3 | 45 |
| | Ppp1r12a | −1.1 | 64.6 | 30.4 |
| | Ptk2 | −1.9 | 9.9 | 2.6 |
| | Tln2 | 2 | 2.2 | 8.8 |
| | Tlr9 | −2.4 | 29.7 | 5.5 |
| | Tspan2 | −3.1 | 11.8 | 1.3 |
| | Tspan4 | 1.1 | 9.7 | 21.4 |
| | Tspan7 | −1.3 | 1.1 | 0.4 |
| ERK/MAPK | Esr1 | −1 | 29 | 14.2 |
| Signaling | Fgfr1 | −4.1 | 34.2 | 2 |
| (16/191 genes) | Fos | 2.2 | 15.6 | 74.3 |
| | Fyn | −3.1 | 62.3 | 7.1 |
| | Mras | −1.6 | 78.1 | 26.2 |
| | Pak1 | −1.5 | 15.8 | 5.6 |
| | Pik3cg | −1.3 | 110.3 | 45 |
| | Pparg | 1.5 | 18.2 | 52.4 |
| | Ppm1j | −1.6 | 4.4 | 1.4 |
| | Ppp1r14a | −3.5 | 7.4 | 0.6 |
| | Prkar2a | −1.2 | 71.1 | 31.8 |
| | Prkcb | −1.3 | 107.7 | 43.6 |
| | Prkcg | −1.1 | 3.2 | 1.4 |
| | Plk2 | −1.9 | 9.9 | 2.6 |
| | Tln2 | 2 | 2.2 | 8.8 |
| | Tlr9 | −2.4 | 29.7 | 5.5 |

^{a}Transcripts per Million

These results demonstrate that, using a neoepitope tumor rejection antigen, macrophages are not effective adjuvants, but DCs are. GM-CSF-BMDCs, FLT3L-BMDCs, and Mo-DCs are all excellent adjuvants, although the GM-CSF DCs seem to be more effective. GM-CSF-BMDCs have been previously characterized as a heterogeneous population consisting ofun-differentiated cells, DC-like cells as well as macrophage-like cells. In the experiments described above, it was observed that this heterogeneous population consists of cells with cell surface markers of DCs as well as macrophage without a clear demarcation between DC-like and macrophage-like cells. Instead the heterogeneity observed is in the maturation status of these DCs. One major sub-population, P5, is more akin to mature DCs, while the P6 sub-population is similar to immature DCs. It is possible that differences in culture conditions (GM-CSF alone in this study as compared with GM-CSF/IL-4 in the study of Helft et al.) are responsible for the differences. Interestingly, it was observed in the experiments described above that while both P5 and P6 sub-populations are effective adjuvants, the P6 sub-population is clearly more effective than the P5. The immature DC phenotype of the P6 sub-population, with a higher capacity for antigen uptake, and possibly a higher antigen sequestering capacity, may be responsible for this superior activity. DCs were previously demonstrated to have a unique ability to sequester antigenic epitopes or their precursors for extended periods of time, up to several weeks. Without being bound by a particular theory, it is possible that the P6 sub-population has a better antigenic sequestering ability than the P5. In terms of transcriptional profiles as well, the P6 sub-population expresses higher levels of a variety of immunologically important receptors including heat shock protein receptors CD91 and LOX1, mannose receptors as well as selected TLRs.

These results also provide insights into the mechanisms by which exogenous DCs mediate CD8 immunity. Previous studies have described that DCs-as-adjuvants act as ADCs only or APCs only. Using two distinct methods of analysis, the results described in the experiments above show clearly that DCs act in both capacities, although un-equally. The major contribution towards the adjuvanticity of DCs derives from their role as ADCs, possibly because of the unusual capacity of DCs to sequester antigen for prolonged periods of time. The identification of a specific sub-set of DCs with the highest adjuvanticity as well as a better understanding of the mechanisms of their adjuvanticity allows the use DCs as highly effective adjuvants, for example, in neoepitope-based cancer vaccines.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

Incorporation by reference: Various references such as patents, patent applications, and publications are cited herein, the disclosures of which are hereby incorporated by reference herein in their entireties.

1. An isolated CD11c⁺ MHCII^{lo} CD11b^{hi}, CD24^{lo}, CD40⁻, CD86^{lo} dendritic cell.

2. A composition comprising
an isolated CD11c⁺ MHCII^{lo} CD11b^{hi}, CD24^{lo}, CD40⁻, CD86^{lo} dendritic cell derived from the blood or bone marrow of a cancer subject, and
a neoepitope peptide or nucleic acid molecule encoding the neoepitope peptide, wherein the neoepitope peptide is specific to a tumor from the cancer patient.

3. The composition of claim 2, wherein the neoepitope peptide is not from known cancer-causing pathways.

4. The composition of any one of claims 2 and 3, wherein the neoepitope peptide has a conformational stability when bound to an MHCI or MHC II protein as determined by molecular modeling or experiment that is higher compared to the corresponding wild type epitope.

5. The composition of any one of claims 2-4, wherein the neoepitope peptide is identified by the method of US2015/0252427 or WO 2016/040110.

6. The composition of any one of claims 2-5, further comprising an immune-modulating agent.

7. The composition of claim 6, wherein the immune-modulating agent is an anti-cytotoxic T-lymphocyte antigen-4 antibody (anti-CTLA-4).

8. The composition of any one of claims 1-7, wherein the composition further comprises an adjuvant.

9. An immunotherapy method comprises administering the composition of any one or more of claims 2-8 to the cancer patient.

10. The method of claim 9, further comprising treating the cancer patient with radiation therapy, chemotherapy, surgery, or a combination comprising one or more of the foregoing.

11. A method of producing an immunotherapeutic composition comprises
isolating monocytes from a cancer patient's peripheral blood mononuclear cells or obtaining bone marrow cells from the cancer patient,
differentiating the monocytes or the bone marrow cells toward dendritic cells using GM-CSF, IL-4, or both,
FACS sorting the differentiated cells based on MHCII and D11c expression, and
isolating from the sorted cells a population of cells with the same phenotype as a mouse CD11c⁺ MHCII^{lo} CD11b^{hi}, CD24^{lo}, CD40⁻, CD86^{lo} GM-CSF derived dendritic cell, and
pulsing the CD11c⁺ MHCII^{lo} CD11b^{hi}, CD24^{lo}, CD40⁻, CD86^{lo} dendritic cells with a patient tumor neoepitope peptide or a nucleic acid encoding a neoepitope peptide to provide the immunotherapeutic composition.

12. The method of claim 11, wherein the neoepitope peptide is not from known cancer-causing pathways.

13. The method of any one or more of claims 11 and 12, wherein the neoepitope peptide has a conformational stability when bound to an MHCI or MHC II protein as determined by molecular modeling or experiment is higher compared to the corresponding wild type epitope.

14. The method of any one of claims 11-13, wherein the neoepitope peptide is identified by the method of US2015/0252427 or WO 2016/040110.

15. The method of any one or more of claims 11-14, further comprising administering an immune-modulating agent.

16. The method of claim 15, wherein the immune-modulating agent is an anti-cytotoxic T-lymphocyte antigen-4 antibody (anti-CTLA-4).

17. An isolated CD11c⁺ MHCII^{lo/int} CD11b^{hi}, CD24^{-/lo}, CD40^{-/lo}, CD86^{-/lo} dendritic cell.

18. A composition comprising
an isolated CD11c⁺ MHCII^{lo/int} CD11b^{hi}, CD24^{-/lo}, CD40^{-/lo}, CD86^{-/lo} dendritic cell derived from the blood or bone marrow of a subject, and
a neoepitope peptide or nucleic acid molecule encoding the neoepitope peptide, wherein the neoepitope peptide is specific to a tumor from a cancer patient, wherein the subject is a healthy subject or the cancer patient.

19. The composition of claim 18, wherein the subject is the cancer patient.

20. The composition of claim 18 or 19, wherein the neoepitope peptide is not from known cancer-causing pathways.

21. The composition of any one of claims 18-20, wherein the neoepitope peptide has a higher conformational stability when bound to an MHCI or MHC II protein compared to that of the corresponding wild type epitope.

22. The composition of any one of claims 18-21, further comprising an immune-modulating agent.

23. The composition of claim 22, wherein the immune-modulating agent is an anti-cytotoxic T-lymphocyte antigen-4 antibody (anti-CTLA-4).

24. The composition of any one of claims 18-23, wherein the composition further comprises an adjuvant.

25. An immunotherapy method comprises administering the composition of any one of claims 18-24 to the cancer patient.

26. The method of claim 25, further comprising treating the cancer patient with radiation therapy, chemotherapy, surgery, or a combination comprising one or more of the foregoing.

27. A method of producing an immunotherapeutic composition comprises
isolating monocytes from a subject's peripheral blood mononuclear cells or obtaining bone marrow cells from the subject,
differentiating the monocytes or the bone marrow cells toward dendritic cells,
FACS sorting the differentiated cells based on MHCII and CD11c expression, and
isolating from the sorted cells a population of CD11c⁺ MHCII^{lo/int} CD11b^{hi}, CD24^{-/lo}, CD40^{-/lo}, CD86^{-/lo} dendritic cell, and
pulsing the CD11c⁺ MHCII^{lo/int} CD11b^{hi}, CD24^{-/lo}, CD40^{-/lo}, CD86^{-/lo} dendritic cells with a patient tumor neoepitope peptide or a nucleic acid encoding a neoepitope peptide from a cancer patient to provide the immunotherapeutic composition, wherein the subject is a healthy subject or the cancer patient.

28. The method of claim 27, wherein the subject is the cancer patient.

29. The method of claim 27 or 28, wherein the neoepitope peptide is not from known cancer-causing pathways.

30. The method of any one of claims 27-29, wherein the neoepitope peptide has a higher conformational stability when bound to an MHCI or MHC II protein compared to that of the corresponding wild type epitope.

31. The method of any one of claims 27-30, further comprising administering an immune-modulating agent.

32. The method of claim 31, wherein the immune-modulating agent is an anti-cytotoxic T-lymphocyte antigen-4 antibody (anti-CTLA-4).

33. A method of preparing a therapeutic composition, comprising combining one or more neoepitope peptides with the cell of claim 1 or 17.

34. The method of claim 33, wherein the combining with the cell comprises pulsing of the cell with the neoepitope peptides.

35. The method of claim 33 or 34, wherein the neoepitope peptides are synthesized prior to the combining with the cell.

36. The method of claim 33, 34 or 35, wherein the neoepitope peptides are identified using Differential Agretopic Index (DAI).

37. The method of any one of claim 33-36, wherein the neoepitope peptides are identified by assessing conformational stability of the neoepitope peptides when bound to an MHCI or MHC II protein.

38. The method of any one of claims 27-32, wherein the differentiating step is performed using GM-CSF, IL-4, or both GM-CSF and IL-4.

39. An isolated population of CD11c⁺ MHCII^{lo} CD11b^{hi}, CD24^{lo}, CD40⁻, CD86^{lo} dendritic cells.

40. An isolated population of CD11c⁺ MHCII^{lo/int} CD11b^{hi}, CD24^{-/lo}, CD40^{-/lo} CD86^{-/lo} dendritic cells.

41. A composition comprising
the isolated population of dendritic cells of claim 39, derived from the blood or bone marrow of a subject, and
a neoepitope peptide or nucleic acid molecule encoding the neoepitope peptide,
wherein the neoepitope peptide is specific to a tumor from a cancer patient, wherein
the subject is a healthy subject or the cancer patient.

42. A composition comprising
the isolated population of dendritic cells of claim 40, derived from the blood or bone marrow of a subject, and
a neoepitope peptide or nucleic acid molecule encoding the neoepitope peptide,
wherein the neoepitope peptide is specific to a tumor from a cancer patient, wherein
the subject is a healthy subject or the cancer patient.

43. The composition of claim 41, wherein the subject is the cancer patient.

44. The composition of claim 42, wherein the subject is the cancer patient.

45. An immunotherapy method comprising administering the composition of any one of claims 41-44 to the cancer patient.

46. An isolated population of dendritic cells expressing as indicated:
(a) at least 1, at least 2, or least 3 of the following markers: CD91^{hi}, LOX1^{hi}, TLR2^{hi}, CD80^{-/lo}, CD36^{lo}, CD209a^{-/lo}, mannose receptor C-type 1^{hi}, macrophage scavenger receptor 1^{hi}, TLR1^{hi}, TLR6^{hi}, and TLR7^{hi}; or
(b) at least 3 of the following markers: CD91^{hi}, LOX1^{hi}, TLR2^{hi}, CD80^{-/lo}, CD36^{lo}, CD209a^{-/lo}, mannose receptor C-type 1^{hi}, macrophage scavenger receptor 1^{hi}, TLR1^{hi}, TLR6^{hi}, TLR7^{hi}, C1s1^{hi}, Cfb^{hi}, Fos^{hi}, Hp^{hi}, Il18^{hi}, Il1a^{hi}, Il1f9^{hi}, Serpine1^{hi}, Serpinf1^{lo}, Fos^{hi}, Il18^{hi}, Il1a^{hi}, Il1f9^{hi}, Il1rl1^{lo}, Ccr7^{lo}, Cd40^{lo}, Cd83^{lo}, Cd86, Fgfr1^{lo}, Fscn1^{lo}, H2-Q6^{lo}, H2-DMb2^{lo}, H2-Oa^{lo}, H2-Ob^{lo}, H2-Aa^{lo}, H2-Ab1^{lo}, H2-Ea-ps^{lo}, H2-Eb1^{lo}, Il18^{hi}, Il1a^{hi}, Il1f9^{hi}, Jak2, Lepr, Jak2^{lo}, Stat4^{lo}, Fyn^{lo}, Itgae^{lo}, Mylk^{lo}, Ptk2^{lo}, Tln2^{hi}, Tlr9^{lo}, Tspan2^{lo}, Fos^{hi}, and Ppp1r14a^{lo}.

47. The isolated population of dendritic cells of claim 46, further expressing as indicated the following markers: CD11c⁺, MHCII^{lo/int}, and CD11b^{lo}.

48. The isolated population of dendritic cells of claim 46 or 47, further expressing as indicated the following markers: CD24^{-/lo}, CD40^{-/lo}, and CD86^{-/lo}.

49. A composition comprising
the isolated population of dendritic cells of any one of claims 46-48, derived from the blood or bone marrow of a subject, and
a neoepitope peptide or nucleic acid molecule encoding the neoepitope peptide,
wherein the neoepitope peptide is specific to a tumor from a cancer patient, wherein
the subject is a healthy subject or the cancer patient.

50. The composition of claim 49, wherein the subject is the cancer patient.

51. An immunotherapy method comprises administering the composition of claim 49 or 50 to the cancer patient.

52. A method of producing an immunotherapeutic composition comprising:
(i) isolating monocytes from a subject's peripheral blood mononuclear cells or obtaining bone marrow cells from the subject,
(ii) differentiating the monocytes or the bone marrow cells toward dendritic cells, and
(iii) isolating from the differentiated cells a population of cells expressing:
(a) at least 1, at least 2, or least 3 of the following markers as indicated: CD91^{hi}, LOX1^{hi}, TLR2^{hi}, CD80^{-/lo}, CD36^{lo}, CD209a^{-/lo}, mannose receptor C-type 1^{hi}, macrophage scavenger receptor 1^{hi}, TLR1^{hi}, TLR6^{hi}, and TLR7^{hi}; or
(b) at least 3 of the following markers as indicated: CD91^{hi}, LOX1^{hi}, TLR2^{hi}, CD80^{-/lo}, CD36^{lo}, CD209a^{-/lo}, mannose receptor C-type 1^{hi}, macrophage scavenger receptor 1^{hi}, TLR1^{hi}, TLR6^{hi}, TLR7^{hi}, C1s1^{hi}, Cfb^{hi}, Fos^{hi}, Hp^{hi}, Il18^{hi}, Il1a^{hi}, Il1f9^{hi}, Serpine1^{hi}, Serpinf1^{lo}, Il18^{hi}, Il1a^{hi}, Il1f9^{hi}, Il1rl1^{lo}, Ccr7^{lo}, Cd40^{lo}, Cd83^{lo}, Fgfr1^{lo}, Fscn1^{lo}, H2-DMb2^{lo}, H2-Oa^{lo}, H2-Ob^{lo}, H2-Aa^{lo}, H2-Ab1^{lo}, H2-Ea-ps^{lo}, H2-Eb1^{lo}, Il18^{hi}, Il1a^{hi}, Il1f9^{hi}, Jak2^{lo}, Stat4^{lo}, Fyn^{lo}, Itgae^{lo}, Mylk^{lo}, Ptk2^{lo}, Tln2^{hi}, Tlr9^{lo}, Tspan2^{lo}, and Ppp1r14a^{lo}, and
(iv) combining the isolated dendritic cells with a patient tumor neoepitope peptide or a nucleic acid encoding a neoepitope peptide from a cancer patient to provide the immunotherapeutic composition, wherein the subject is a healthy subject or the cancer patient.

53. The method of claim 52, wherein the subject is the cancer patient.

54. The method of claim 52 or 53, wherein the population of cells isolated in step (iii) further expresses the following markers as indicated: CD11c⁺, MHCII^{lo/int}, and CD11b^{hi}.

55. The method of any one of claims 52-54, wherein the population of cells isolated in step (iii) further expresses the following markers as indicated: CD24^{-/lo}, CD40^{-/lo}, and CD86^{-/lo}.

56. A method of preparing a therapeutic composition, comprising combining one or more neoepitope peptides with the isolated population of cells of any one of claims 46-48.


SEQUENCE LISTING (3 sequences)

SEQ ID NO: 1 - protein, 8 aa, Artificial Sequence
  epitope peptide
SIINFEKL

SEQ ID NO: 2 - protein, 9 aa, Artificial Sequence
  epitope peptide
SSIINFEHL

SEQ ID NO: 3 - protein, 18 aa, Artificial Sequence
  epitope peptide
LEQLKSIINFEHLKEWTS